(19) United States
(12) Patent Application Publication
(10) Pub. No.: US 2022/0172812 A1
(43) Pub. Date: Jun. 2, 2022

(54) PK/PD Prediction Using an Ode-Based Neural Network System

(71) Applicant: Genentech, Inc., South San Francisco, CA (US)

(72) Inventor: James LU, San Francisco, CA (US)

(21) Appl. No.: 17/468,636

(22) Filed: Sep. 7, 2021

Related U.S. Application Data
(60) Provisional application No. 63/075,793, filed on Sep. 8, 2020.
(60) Provisional application No. 63/104,342, filed on Oct. 22, 2020.
(60) Provisional application No. 63/164,505, filed on Mar. 22, 2021.
(60) Provisional application No. 63/185,962, filed on May 7, 2021.

(51) Int. Cl. G16H 20/10; G16H 50/20

(52) U.S. Cl. CPC G16H 20/10; G16H 50/20

(57) ABSTRACT

A method for predicting pharmacokinetic-pharmacodynamic effects over time is provided. A pharmacokinetic pathway of a neural network system that lies at least partially within an ordinary differential equations (ODE) module of the neural network system is trained to generate a dose effect output associated with a drug. A pharmacodynamic pathway of the neural network system that lies at least partially within the ODE module is trained to generate a drug effect output associated with the drug. The drug effect output associated with an administration of the drug over a time period is predicted using the neural network system.

### CROSS-REFERENCE TO RELATED APPLICATION

This provisional application claims priority to U.S. Provisional Patent Application No. 63/185,962, filed May 7, 2021, U.S. Provisional Patent Application No. 63/164,505, filed Mar. 22, 2021, U.S. Provisional Patent Application No. 63/104,342, filed Oct. 22, 2020, and U.S. Provisional Patent Application No. 63/075,793, filed Sep. 8, 2020, each of which is incorporated herein by reference in its entirety.

### FIELD

This description is generally directed towards systems and methods for predicting (or estimating) pharmacological properties of drugs (e.g., therapeutics). More specifically, machine learning-based systems and methods for accurately predicting pharmacokinetic and pharmacodynamic effects using an ordinary differential equations (ODE) neural network are disclosed herein.

### BACKGROUND

The development of new drugs (e.g., therapeutics) is driven by progress in many disciplines. Such disciplines include drug discovery, biotechnology, and in vivo and in vitro pharmacological/toxicological characterization techniques. Before a new therapeutic can move from a molecule or protein in the laboratory to become a new product in the hospital/clinic or local pharmacy, various questions must be answered with respect to the efficacy, administration, safety, and side effects associated with the therapeutic. Answering these types of questions typically involves a series of clinical trials, which are carefully designed to study the various facets of a new drug candidate.

Pharmacokinetics (PK) and pharmacodynamics (PD) are scientific disciplines associated with therapeutic development that typically involve mathematical modeling. In popular terms, PK is often described as “what the body does to the drug” and PD as “what the drug does to the body.” More specifically, PK focuses on modeling how the body acts on the drug once it is administered and is subjected to the four bodily processes of absorption, distribution, metabolism and elimination or excretion (ADME). Often, this is accomplished by modeling concentrations in the body generally or in various areas of the body as a function of time. PD aims at linking these modeled drug concentrations to certain drug effects through a PD-model specifically designed to evaluate those effects. PK/PD modeling is thus a discipline that was developed to link systemic drug concentration kinetics to the resulting drug effects over time. Such modeling enables the description and prediction of the time course of various physiological effects (e.g., tumor cell count, platelet count, neutrophil count, etc.) in response to various dosage regimens.

Conventional mathematical modeling methodologies for PK/PD evaluation typically require iterations of model evaluation and refinement, with human judgement involved in various steps within the loop. This can be time and labor intensive. Examples of such existing mathematical algorithms include expectation-maximization, genetic algorithms, and scatter search. These techniques may be optimization-based, which in practice may mean that the scientist creating the model performs many function and gradient evaluations involving significant trial-and-error. Accordingly, effectively using these existing mathematical techniques to model PK and PD involves a significant amount of know-how and computational time. The know-how prerequisite and computational resource requirement represent significant obstacles along the path towards the broad adoption of PK, PD, and PK/PD modeling for non-expert users.

### SUMMARY

In various embodiments, a method is provided for predicting pharmacokinetic-pharmacodynamic effects over time. A pharmacokinetic pathway of a neural network system that lies at least partially within an ordinary differential equations (ODE) module of the neural network system is trained to generate a dose effect output associated with a drug. A pharmacodynamic pathway of the neural network system that lies at least partially within the ODE module is trained to generate a drug effect output associated with the drug. A drug effect of an administration of the drug to a subject over a time period is predicted by generating the drug effect output suing using the neural network system having the trained pharmacokinetic pathway and the trained pharmacodynamic pathway.

In various embodiments, a non-transitory computer-readable medium storing computer instructions for predicting pharmacokinetic-pharmacodynamic effects over time is provided. The non-transitory computer-readable medium comprises machine-executable code which, when executed by at least one machine, causes the at least one machine to train a pharmacokinetic pathway of a neural network system that lies at least partially within an ordinary differential equations (ODE) module of the neural network system to generate a dose effect output associated with a drug. The machine-executable code, when executed by at least one machine, further causes the at least one machine to train a pharmacodynamic pathway of the neural network system that lies at least partially within the ODE module to generate a drug effect output associated with the drug. The machine-executable code, when executed by at least one machine, further causes the at least one machine to predict the drug effect output associated with an administration of the drug over a time period using the neural network system.

In various embodiments, a system is provided for predicting pharmacokinetic-pharmacodynamic effects over time. The system comprises a memory containing machine readable medium comprising machine executable code and a processor coupled to the memory. The processors is configured to execute the machine executable code to cause the processor to train a pharmacokinetic pathway of a neural network system that lies at least partially within an ordinary differential equations (ODE) module of the neural network system to generate a dose effect output associated with a drug; train a pharmacodynamic pathway of the neural network system that lies at least partially within the ODE module to generate a drug effect output associated with the drug; and predict the drug effect output associated with an administration of the drug over a time period using the neural network system.

In various embodiments, a method is provided for training a pharmacokinetic/pharmacodynamic neural network system. Training data is provided. The training data includes measured dose effect and measured drug effect over an initial time period. A pharmacokinetic pathway of a neural network system is trained using a first portion of the training data to form a trained pharmacokinetic encoder and a trained pharmacokinetic submodule of an ordinary differential equations (ODE) module in the neural network system. A pharmacodynamic pathway of the neural network system is trained using a second portion of the training data and an initial condition pathway of the neural network system using a third portion of the training data with the trained pharmacokinetic encoder and the trained pharmacokinetic submodule fixed to thereby form a trained pharmacodynamic encoder, a trained pharmacodynamic submodule of the ODE module, and a trained initial condition submodule. The trained pharmacokinetic submodule generates a dose effect output and the trained pharmacodynamic submodule generates a drug effect output.

In various embodiments, a method is provided for predicting pharmacokinetic-pharmacodynamic effects over time. Initial subject data is received for an initial time period. A pharmacokinetic vector is generated based on a first portion of the initial subject data using a pharmacokinetic encoder. A pharmacodynamic vector is generated based on a second portion of the initial subject data using a pharmacodynamic encoder. A dose effect output is predicted based on the pharmacokinetic vector, dose amount data, and an initial condition using an ordinary differential equations (ODE) module. A drug effect output is predicted based on the dose effect output, the pharmacodynamic vector, and an initial condition using the ODE module.

In various embodiments, a non-transitory computer-readable medium storing computer instructions for training a neural network system is provided. The non-transitory computer-readable medium comprises machine-executable code which, when executed by at least one machine, causes the at least one machine to provide, by one or more processors, training data to the neural network system, wherein the training data includes measured dose effect and measured drug effect over an initial time period. The machine-executable code, when executed by at least one machine, further causes the at least one machine to train, by the one or more processors, a pharmacokinetic pathway of a neural network system using a first portion of the training data to form a trained pharmacokinetic encoder and a trained pharmacokinetic submodule of an ordinary differential equations (ODE) module in the neural network system. The machine-executable code, when executed by at least one machine, further causes the at least one machine to train, by the one or more processors, a pharmacodynamic pathway of the neural network system using a second portion of the training data and an initial condition pathway of the neural network system using a third portion of the training data with the trained pharmacokinetic encoder and the trained pharmacokinetic submodule fixed to thereby form a trained pharmacodynamic encoder, a trained pharmacodynamic submodule of the ODE module, and a trained initial condition submodule. The trained pharmacokinetic submodule generates a dose effect output and the trained pharmacodynamic submodule generates a drug effect output.

In various embodiments, a non-transitory computer-readable medium storing computer instructions for predicting pharmacokinetic-pharmacodynamic effects over time. The non-transitory computer-readable medium comprises machine-executable code which, when executed by at least one machine, causes the at least one machine to receive, by one or more processors, initial subject data for an initial time period; generate, by the one or more processors, a pharmacokinetic vector based on a first portion of the initial subject data using a pharmacokinetic encoder; generate, by the one or more processors, a pharmacodynamic vector based on a second portion of the initial subject data using a pharmacodynamic encoder; predict, by the one or more processors, a dose effect output based on the pharmacokinetic vector, dose amount data, and an initial condition using an ordinary differential equations (ODE) module; and predict, by the one or more processors, a drug effect output based on the dose effect output, the pharmacodynamic vector, and an initial condition using the ODE module.

### BRIEF DESCRIPTION OF THE DRAWINGS

For a more complete understanding of the principles disclosed herein, and the advantages thereof, reference is now made to the following descriptions taken in conjunction with the accompanying drawings, in which:

FIG. 1 is a block diagram of a pharmacokinetic/pharmacodynamic (PK/PD) evaluation system in accordance with one or more example embodiments.

FIG. 2 is a schematic diagram of a neural network system in accordance with various embodiments.

FIG. 3 is a schematic diagram of the internal architecture of the ODE module from FIG. 2 in accordance with various embodiments.

FIG. 4 is a schematic diagram of the PK submodule from FIG. 3 in accordance with various embodiments.

FIG. 5 is a different schematic diagram of the PK submodule in accordance with various embodiments.

FIG. 6 is a schematic diagram of the PD submodule from FIG. 3 in accordance with various embodiments.

FIG. 7 is a portion of a table of values used as input data in accordance with various embodiments.

FIG. 8 is a flowchart of a process for training a neural network system and using the trained neural network system to predict pharmacokinetic-pharmacodynamic effects over time in accordance with various embodiments.

FIG. 9 is a flowchart of a process for predicting pharmacokinetic-pharmacodynamic effects over time in accordance with various embodiments.

FIG. 10 is a flowchart of a process for training a neural network system to predict pharmacokinetic-pharmacodynamic effects over time in accordance with various embodiments.

FIG. 11 is a block diagram of a computer system in accordance with various embodiments.

FIGS. 12A-12F are plots in a plot series demonstrating the accuracy of the dose effect output of a neural network system in accordance with various embodiments.

FIGS. 13A-13F are plots in a plot series demonstrating the accuracy of the drug effect output of a neural network system in accordance with various embodiments.

FIGS. 14A-14F are plots in another plot series demonstrating the accuracy of the neural network system in accordance with various embodiments.

FIG. 15 is a table comparing the predictive performance of a population PK/PD model to a neural network system per the various embodiments described herein.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

### I. Overview

The principles of pharmacokinetics/pharmacodynamics (PK/PD) have become a well-established quantitative framework for understanding the dose-concentration-effect relationships of various therapeutics and selecting the proper protocols (e.g., dosage, schedules, etc.) for such therapeutics. In particular, the methodology of population PK/PD (pop-PK/PD) modeling has become the “gold-standard” in the longitudinal analysis of subject (or patient) data.

Currently, various PK/PD models are built using ordinary differential equations (ODEs) created by humans. In other words, ODE construction has relied upon human modelers' understanding of dynamical systems and creativity in coming up with the governing equations that can encapsulate the qualitative characteristics observed in the data. Parameter estimation for the system of ODEs may be performed based on assumed statistical distributions of parameters and error models. For example, parameter estimation may be computationally performed using iterative optimization techniques to minimize the discrepancy between an observed and predicted trajectory for a selected error metric. The performance of alternative PK/PD models can be compared, and a selection may be made based on various diagnostic criteria. This type of modeling paradigm involves iterative refinement, and the accuracy of such models for making temporal predictions depends on the human modeler's ability to abstract insights from complex data sets. The range of data modalities in modern biomedical applications includes data from imaging, high dimensional assays, and continuous monitoring devices. Manually extracting insights from the data for these biomedical applications is ever more challenging for human modelers, especially across the range of data modalities.

Recognizing and taking into account the above-described issues, the embodiments described herein provide a novel neural PK/PD modeling framework, based on a recurrent neural network architecture, that combines the principles of PK/PD with deep learning. In particular, the embodiments described herein incorporate principles of PK/PD with neural network-derived ODEs to build PK/PD models. PK/PD models built from the embodiments described herein capture the benefits of directly learning governing equations from input data, while ensuring the fundamental dose-concentration-effect relationship is preserved. For example, using this type of neural PK/PD modeling framework does not require labor intensive testing of alternate model structures or performing diagnostics to compare results in order to arrive at an accurate PK/PD model, as would be the case for human-generated models. Incorporating the fundamental PK/PD relationship directly into the neural PK/PD modeling framework ensures that the PK/PD model has the ability to generalize from existing data and simulate unseen novel doses and dosing frequencies. In this manner, the embodiments described herein permit the crucial transfer of existing PK/PD scientific knowledge into the deep learning paradigm, to ensure the generalizability of the deep learning models for predicting unseen doses or dosing regimens.

As a proof-of-concept of the disclosed methodology, the PK/PD model built using the neural PK/PD modeling framework described herein was applied to a legacy clinical trial data set for over 600 subjects (or patients). This demonstration showed that the neural PK/PD modeling framework can directly learn the system dynamics (e.g., time delay in response, hysteresis behavior, etc.) from input data and build a PK/PD model that numerically improves upon or otherwise outperforms well-established pop-PK/PD modeling techniques with respect to certain prediction performance metrics (e.g., r-squared values between the framework's prediction and the unseen data, the root-mean-squared error for the prediction, etc.).

These results demonstrate the potential of neural PK/PD modeling for enabling automated predictive analytics built upon the foundation of PK/PD. For example, in a clinical development context, the neural PK/PD modeling methodology described herein may enable automated predictive analysis in real-time of subject data from a Phase I study of a therapeutic for use in informing the appropriate dosing regimen to be tested in the Phase II study. In a personalized dosing application, real-time, personalized predictions for the subject may be made based on data measured for that subject. Thus, the neural PK/PD modeling described herein may enable automated predictive analysis of personalized dosing simulations and scheduling and novel dosing regimens as well as the generation of real-time dosing regimens or adjustments.

Although the systems and methods disclosed herein refer to their application in pharmacokinetics (PK) and pharmacodynamics (PD) specifically, it should be appreciated that they are equally applicable to other analogous fields such as, but not limited to, toxicokinetics and toxicodynamics.

### II. PK/PD Modeling Using an ODE-Based Neural Network System

IIA. PK/PD Evaluation System

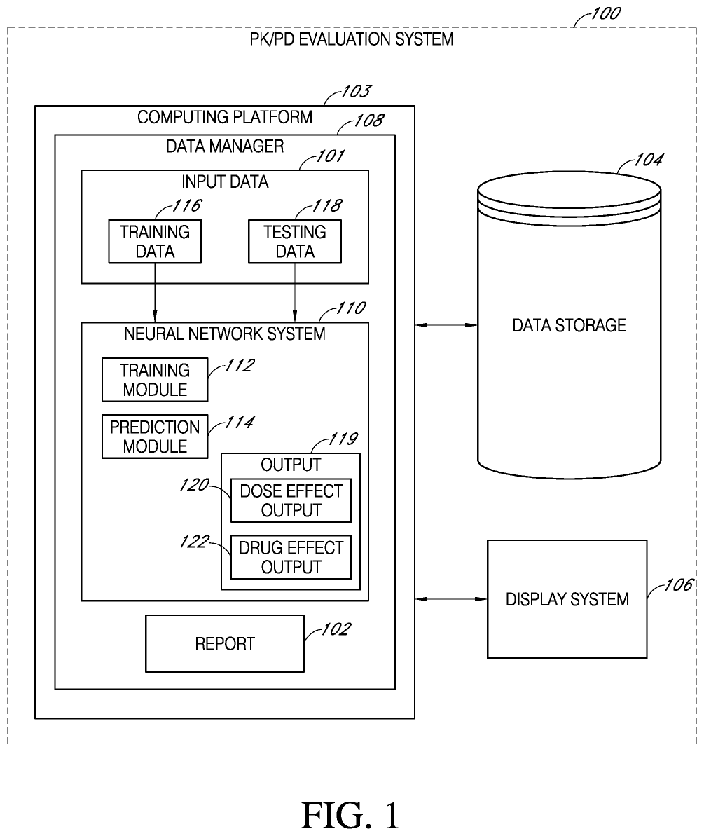

FIG. 1 is a block diagram of a pharmacokinetic/pharmacodynamic (PK/PD) evaluation system 100 in accordance with one or more example embodiments. PK/PD evaluation system 100 may be used to evaluate PK and PD effects resulting from the administration of a drug (e.g., therapeutic). In various embodiments, PK/PD evaluation system 100 is trained based on observed data and then used to predict PK and/or PD effects over time (including time beyond that for which observed data is provided or available). As previously described, a PK effect is a dose effect or an effect of the body on the drug in the body (e.g., drug concentration). Further, a PD effect is an effect of the drug on the body. This effect of the drug on the body is measured using a measurement of a biomarker (e.g., platelets, tumor cells, neutrophils, etc.).

PK/PD evaluation system 100 may be used in various settings including, but not limited to, a clinical trial setting, a drug development setting, a hospital setting, or in some other type of setting. PK/PD evaluation system 100 may receive and process input data 101 to generate a report 102 that describes and/or contains information based on these PK and PD effects. Input data 101 may include, for example, lab measurements taken from the plasma of subjects, measurements from continuous monitoring devices (e.g., in hospital or home settings), or some other type of data. The report 102 may include, for example, at least one of a PK time course or a PD time course that is predicted for a certain number of days or weeks into the future based on a current dosing regimen. In some cases, report 102 may also include at least one of a corresponding PK time course or a corresponding PD time course, respectively, that is predicted based on certain dosing interruptions and/or adjustments to the current dosing regimen. Report 102 may be generated for an individual subject in question or for the entire cohort of subjects in a clinical trial. In one or more examples, report 102 may include one or more recommended actions based on a predicted PK time course, a predicted PD time course, or both.

PK/PD evaluation system 100 includes computing platform 103, data storage 104, and display system 106. Computing platform 103 may take various forms. In one or more embodiments, computing platform 103 includes a single computer (or computer system) or multiple computers in communication with each other. In other examples, computing platform 103 takes the form of a cloud computing platform.

Data storage 104 and display system 106 are each in communication with computing platform 103. In some examples, data storage 104, display system 106, or both may be considered part of or otherwise integrated with computing platform 103. Thus, in some examples, computing platform 103, data storage 104, and display system 106 may be separate components in communication with each other, but in other examples, some combination of these components may be integrated together.

PK/PD evaluation system 100 includes data manager 108 and neural network system 110 implemented in the computing platform 103. Each of the data manager 108 and the neural network system 110 is implemented using hardware, software, firmware, or a combination thereof. The data manager 108 provides input data 101 to the neural network system 110. This input data 101 may be retrieved from the data storage 104, received from some other source, or a combination thereof.

The neural network system 110 includes a plurality of neural network models, which include a neural-ODE. The neural network system 110 may be used to predict PK effects, PD effects, or both. Accordingly, the neural network system 110 may also referred to as a neural PK/PD model.

The neural network system 110 is trained to build a system of ODEs that can predict PK and PD effects based on input data 101. When the neural network system 110 is being trained, the input data 101 takes the form of training data 116. After training, the neural network system 110 may be used in practice to predict PK effects, PD effects, or both based on input data 101 in the form of testing data 118. Thus, the type of input data 101 provided to the neural network system 110 may take different forms depending on whether the neural network system 110 is in a training mode or in a prediction mode.

For example, the neural network system 110 may include a training module 112 and a prediction module 114. The training module 112 may be used when the neural network system 110 is being trained (e.g., in a training mode). As one example, the training module 112 trains the neural network system 110 using training data 116 received from the data manager 108. The prediction module 114 uses the neural network system 110 for prediction (e.g., in a prediction mode). For example, after the neural network system 110 has been trained, the prediction module 114 may be used in practice to predict PK and PD effects using testing data 118 received from the data manager 108.

The neural network system 110 generates an output 119. The output 119 includes a dose effect (i.e., PK) output 120, a drug effect (i.e., PD) output 122, or both. The dose effect output 120 is a dose effect time course. For example, the dose effect output 120 may be a continuous function of dose effect (e.g., drug concentration in the body or in a target area of the body) over time. The dose effect output 120 may be, for example, drug concentration in plasma (CP) over time. The drug effect output 122 is a drug effect time course. For example, the drug effect output 122 may be a continuous function of drug effect (e.g., biomarker effect) over time. A biomarker effect may be, for example, a number or count of platelets, neutrophils, tumor cells, or some other type of biomarker. It should be noted that the drug effect output 122 is dependent on dose effect, which is dependent on a dose of a drug administered to a subject.

As described above, PK/PD evaluation system 100 may be used to generate a report 102. The report 102 may include, for example, a graphical representation of the dose effect output 120, the drug effect output 122, or both. In some cases, the report 102 may include information based on or derived from the dose effect output 120, the drug effect output 122, or both. This graphical representation may include one or more graphs, one or more tables, one or more text summaries, or a combination thereof. The computing platform 103 may display the report 102 or at least some portion of the report 102 on display system 106. The report 102, or at least one of the dose effect output 120 or the drug effect output 122, may be used by an operator to evaluate a performance of the drug for which this information is generated.

In some embodiments, the report 102 may be used to determine whether a drug is having the intended reaction on the body. In some embodiments, the report 102 may be used to determine whether to adjust a dosing of the drug, or to inform the extent to which a dosing should be adjusted. In some embodiments, the report 102 may be used to determine whether the method of administering the drug is to be changed. In other embodiments, the report 102 may be used to select an appropriate dosing schedule for a particular subject (or patient). In other words, the report 102 may be used to generate a personalized dose and dosing schedule for a given subject.

IIB. Neural Network System within a PK/PD Evaluation System Neural Network

II.B.1. Exemplary Neural Network System

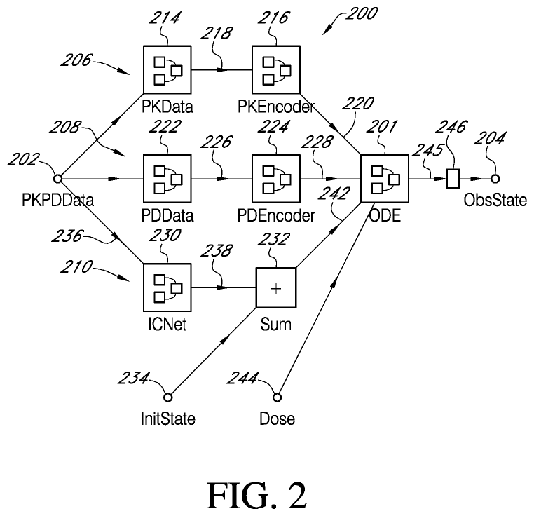

FIG. 2 is a schematic diagram of a neural network system 200 in accordance with various embodiments. The neural network system 200 is one example of an implementation for the neural network system 110 described above with respect to FIG. 1. In particular, the neural network system 200 includes an ordinary differential equation(s) (ODE) module 201 that includes a neural network or neural net.

This ODE module 201 permits automatic selection and generation of ODEs that are used to analyze input data. Such an ODE module 201 removes the need for human expertise and experimentation in determining how to analyze input data, permits novel and multi-faceted analysis that a human may not ascertain, and provides real-time PK/PD analysis of input data. The ODE module 201 is trained to analyze the dose-concentration-effect relationships found within the input data and generate ODEs based on these relationships to determine a predicted PK or PD time course for a given subject.

In addition to the ODE module 201, the neural network system 200 includes various components and neural network models, which may be also referred to as neural networks or neural nets. For example, the neural network system 200 includes input data 202 (e.g., PKPDData), an output 204 (e.g., ObsState), a pharmacokinetic (PK) pathway 206, a pharmacodynamic (PD) pathway 208, and an initial condition (IC) pathway 210. The PK pathway 206 may determine the PK time course (i.e., drug concentrations and corresponding time points). For example, the PK pathway 206 may be used to generate a plot or other relationship of time, relative to drug absorption, distribution, metabolism, and/or excretion. The PD pathway 208 may determine how PD variables change with time in accordance with PK variables. The IC pathway 210 may set the initial condition of the ODE system. Each of the pathways (PK pathway 206, PD pathway 208, and IC pathway 210) may lead to the ODE module 201, which generates output 204. The ODE module 201 may be a recurrent neural net that operates along a time dimension to produce a sequence of predictions for the PK and PD variables at given time intervals. The time intervals may be regular (e.g., equal) time intervals or time intervals of varying length. Each of the PK pathway 206, the PD pathway 208, the IC pathway 210, and the ODE module 201 includes one or more neural networks (or models), each of which is trained such that after training, the neural network system 200 generates a highly accurate and highly reliable output 204.

The neural network system 200 is trained to produce the output 204 based on input data 202, which can include subject data collected for a particular period of time and corresponding to a study, a clinical trial, or another type of experiment. In this manner, input data 202 is an example of one implementation for training data 116 described in FIG. 1. The input data 202 may take various forms. In one or more examples, the input data 202 takes the form of a table of values that may include any number of columns of data that provide information regarding drug dosing, drug concentration (i.e., PK), drug effect (i.e., PD), subject characteristics (e.g., demographic data such as age, sex, disease status, etc.), and baseline data (e.g., baseline values for lab/hematology measurements such as albumin, C-Reactive Protein, blood cell counts, etc.), and time.

The output 204 includes a dose effect output (or PK output), a drug effect output (or PD output), or both. For example, the dose effect output may be one example of an implementation for dose effect output 120 in FIG. 1; the drug effect output may be one example of an implementation for drug effect output 122 in FIG. 1. As previously described, a dose effect output may be a time course that describes a dose effect over time. The dose effect may be, for example, drug concentration in the body (e.g., systemic drug concentration, target area drug concentration), which is dependent on drug dose. A drug effect output may be a drug effect time course that describes the drug effect over time. The drug effect may be, for example, a biomarker effect (e.g., tumor cell count, platelet cell count, neutrophil count, etc.) over time based on drug concentration.

This period of time may be, for example, the length of a clinical trial, the length of one or more stages of a clinical trial, or some other observation window. Further, this period of time may be, for example, 21 days, 42 days, 63 days, or some other number of days. In other examples, this period of time may be in minutes, hours, weeks, months, or some other unit of time.

The PK pathway 206 includes a PK data selector 214 and a PK encoder 216. The PK data selector 214 selects a first portion 218 of the input data 202 for processing and sends this first portion 218 to the PK encoder 216. In the various embodiments, the PK encoder 216 includes a set of gated recurrent units (GRUs). The first portion 218 of the input data 202 may include all or some of the input data 202. In one or more examples, this first portion 218 includes values in the input data 202 that relate to dosing, such as, for example, values for time-after-dose, time, dose effect (PK), and dose amount (or dosage). The PK encoder 216 processes this first portion 218 of the input data 202 and generates a PK vector 220 (e.g., comprised of PK parameters) that is sent to the ODE module 201 for processing.

The PD pathway 208 includes a PD data selector 222 and a PD encoder 224. The PD data selector 222 selects a second portion 226 of the input data 202 for processing and sends this second portion 226 to the PD encoder 224. In the various embodiments, the PD encoder 224 includes a set of GRUs. The second portion 226 of the input data 202 may include all or some of the input data 202. In various embodiments, the second portion 226 is different from the first portion 218 of the input data 202. In one or more examples, this second portion 226 includes values in the input data 202 that relate to drug effect such as, for example, values for time-after-dose, time, dose effect (PK), and drug effect (PD). The PD encoder 224 processes this second portion 226 and generates a PD vector 228 (e.g., comprised of PD parameters) that is sent to the ODE module 201 for processing.

The IC pathway 210 includes an IC submodule 230, a sum submodule 232, and an initial state input 234. The IC submodule 230, which may be an initial condition neural network (e.g., ICNet) receives as input, a third portion 236 of the input data 202. This third portion 236 may include some or all of the input data 202. In one or more examples, this third portion 236 includes values for time-after-dose, time, dose effect (PK), drug effect (PD), and dose amount. In the various embodiments, the IC submodule 230 includes a set of GRUs. The IC submodule 230 processes the third portion 236 of the input data 202 and generates an IC correction 238 to be applied to the initial state input 234.

The initial state input 234 may include, for example, a representation of the dose effect (PK) value and the drug effect (PD) value for an initial point in time per the input data 202. This initial point in time may or may not be where time is equal to zero. For example, the earliest point in time for which the input data 202 includes a drug effect (PD) value may be 2 hours, 6 hours, 24 hours, 2 days, or some other point in time after time zero. The IC correction 238, which is in the form a vector, is summed with the initial state input 234, which is also in the form of a vector, by the sum submodule 232 to produce an IC vector 242 to the ODE module 201. The IC vector 242 represents the best approximated (or actual) initial condition for time equals zero. The portion of the IC correction 238 corresponding to PK may be zero since prior to drug dosing, drug concentration is zero. On the other hand, the drug effect variable (e.g., biomarker) being studied may be non-zero prior to dosing.

The ODE module 201 may comprise a PK/PD vector field (VF). The ODE module 201 receives as input: the PK vector 220 that is output from the PK encoder 216, the PD vector 228 that is output from the PD encoder 224, the IC vector 242 that is output from the sum submodule 232, and a dose input 244. In one or more examples, the dose input 244 is identified from the input data 202. In some cases, the dose input 244 for different time steps is identified from a different table or data set. For example, the dose input 244 may include a dosing desired to be simulated. Using the various inputs, the ODE module 201 generates a set of ordinary differential equations (ODEs), which may also be referred to as a system of ODEs or an ODE system. The ODE module 201 processes the various inputs received using the generated ODE system and generates an ODE output 245 (e.g., vector field (VF) output).

The ODE output 245 includes a representation of dose effect and drug effect in vector form. As one example, the ODE output 245 includes a vector of numeric values (or numbers) for each time step, a first portion (“PK ODE output”) of these numbers which may be converted into a dose effect (PK) value and a second portion (“PD ODE output”) of these numbers which may be converted into a drug effect (PD) value. For example, the neural network system 200 may include a decoder (or converter) 246 that is implemented either outside of or integrated within the ODE module 201. The decoder 246 decodes the ODE output 245, which is a vector, to produce the output 204, which includes a dose effect (PK) value (e.g., drug concentration) and a drug effect (PD) value (e.g., biomarker count or other drug effect value) for each time step. This decoding may be performed using, for example, a set of formulas or equations. In one or more examples, these time steps are constant-sized steps that result in a forward Euler discretization of a continuous ODE system. A time step may be, for example, in minutes, hours, or days. For example, the time step may be one hour, one half of an hour (or 30 minutes), 15 minutes, 2 hours, 3 hours, 4 hours, 6 hours, 12 hours, 24 hours, or some other interval of time. Thus, the ODE module 201 may generate the output 204 including a continuous dose effect (PK) time course and a continuous drug effect (PD) time course.

II.B.2. Exemplary ODE Module Architecture

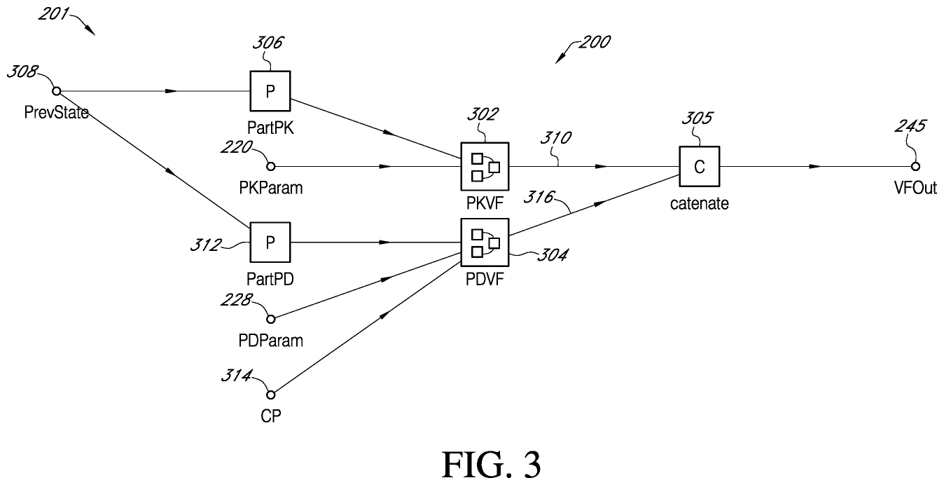

FIG. 3 is a schematic diagram of the internal architecture of the ODE module 201 from FIG. 2 in accordance with various embodiments. The ODE module 201, which is included within the neural network system 200 in FIG. 2, uses at least one neural network to generate a system of equations (i.e., ODEs). The ODE module 201 then uses these generated equations to evaluate and compute dose-concentration-effect relationships that can be derived from input data. The present ODE module 201 generates the equations directly from input data, thus providing an analysis that accounts for the full complexity and range of factors, features, and relationship present in the input data.

In one or more embodiments, the neural networks of ODE module 201 generates the equations based on a combination of the input data and a set of PK/PD principles (e.g., “rules”). Such rules include, but are not limited to: (1) PK is driven by dosing and is independent of PD; (2) the measured drug concentrations versus the total amount in a subject's blood is scaled by a parameter (e.g., the “volume of distribution”; and (3) PD is influenced by both PK and PD, etc. The above rules (1) and (3) are built into the overall architecture of the ODE module 201 via at least the dose input 244 of FIG. 2 and the calculation of the PK and PD vector fields (e.g., 310 and 316, respectively), as shown in FIG. 3.

The training of the ODE module 201 may be performed from a “blank state” (e.g., from a randomized set of initial weights). In other words, the initial state of the ODE module 201 prior to training is a blank state. In other examples, the ODE module 201 is trained from an initial state or default state that is based on a pre-trained network that was previously trained on related PK/PD data sets. Different input PK and/or PD data may result in the ODE module 201 providing different equations (e.g., different ODEs).

The ODE module 201 includes a PK submodule 302, a PD submodule 304, and a catenation unit 305. The PK submodule 302, which comprises a PK vector field (PKVF), receives as input, a previous PK state 306 extracted from a previous state 308 of the ODE output 245 of the ODE module 201 and the PK vector 220 from the PK encoder in FIG. 2. The previous state 308 of the ODE output 245 is the state of the ODE output 245 at the immediately preceding time step. For example, the previous state 308 may be the vector of numeric values generated at the immediately preceding time step. The previous PK state 306 is the portion of this previous state 308 that corresponds to PK or, in other words, the portion used by the decoder 246 in FIG. 2 to generate a dose effect (PK value). For example, the previous state 308 may be a vector of 6 different numeric values, with 2 of those values corresponding to (e.g., used for computing) a PK value and 4 of those values corresponding to (e.g., used for computing) a PD value. Thus, in this example, the previous PK state 306 includes the 2 values corresponding to the PK value. The 2 values may be “decoded” or converted by the decoder 246 in FIG. 2 into the PK value. The PK submodule 302 includes one or more neural networks that use the previous PK state 306 and the PK vector 220 to generate a current PK state 310 for the ODE output 245.

The PD submodule 304, which comprises a PD vector field (PDVF), receives as input, a previous PD state 312 extracted from the previous state 308 of the ODE output 245, the PD vector 228 from the PD encoder 224 in FIG. 2, and the dose effect value 314 for the immediately preceding time step. The previous PD state 312 is the portion of the previous state 308 corresponding to PD. With respect to the example described above, the previous PD state 312 includes the 4 values corresponding to (e.g., used for computing) the PD value. The dose effect value 314 is the decoded or converted form of the previous PK state 306. The decoded or converted form may be, for example, the PK value (e.g., drug concentration) computed by the decoder 246 in FIG. 2 using the portion of the previous state 308 (e.g., the 2 values) corresponding to PK value. In this manner, the dose effect value 314 may be the drug concentration value included in the output 204 described in FIG. 2. The drug concentration value may be, for example, drug concentration in plasma (CP). The PD submodule 304 includes one or more neural networks that use the previous PD state 312 and the PD vector 228 to generate a current PD state 316 for the ODE output 245.

The catenation unit 305 combines the current PK state 310 and the current PD state 316 to form the ODE equations and the ODE output 245. The current state of the ODE output 245 may then be used as the previous state 308 for the next time step.

In various embodiments, the schematic diagram in FIG. 3 is considered a simplified version of the architecture of the ODE module 201. For example, this schematic diagram in FIG. 3 includes at least a portion of the various components (e.g., inputs, logical units, mathematical components, neural network layers, etc.) included in the ODE module 201. In various embodiments, one or more additional components may be included in the ODE module 201. For example, one or more additional components can be used to transform or otherwise process one or more inputs prior to those one or more inputs being sent into the PK submodule 302, the PD submodule 304, or both. As another example, one or more additional components can be used to transform or otherwise process the output of any one or more components in the ODE module 201 prior to forming the final ODE output 245.

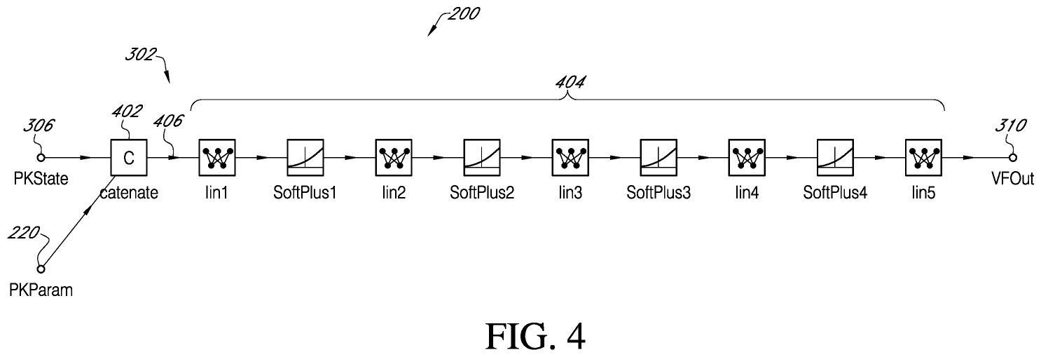

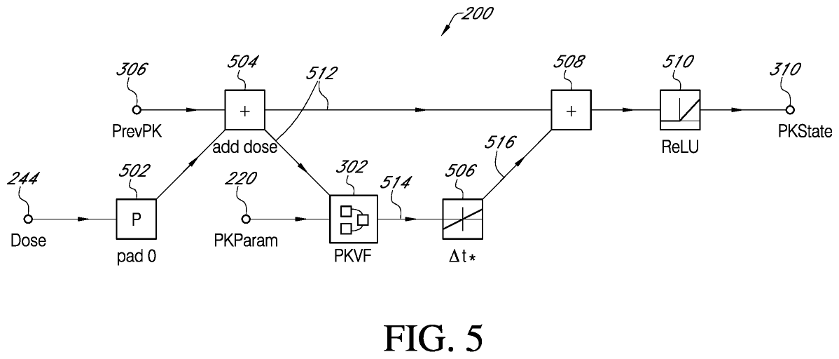

FIG. 4 is a schematic diagram of the PK submodule 302 from FIG. 3 in accordance with various embodiments. The PK submodule 302 is included within the ODE module 201 of the neural network system 200 in FIGS. 2-3. The PK submodule 302 includes a catenation unit 402 and a set of layers 404 that process the output of the catenation unit 402 to then generate the current PK state 310. The catenation unit 402 combines the previous PK state 306 with the PK vector 220 to generate a PK processing vector 406 that is transformed via the set of layers 404. The set of layers 404 may include any number or type of layers including, but not limited to, linear layers and SoftPlus layers. In one or more examples, the set of layers 404 includes 5 linear layers and 5 SoftPlus layers as shown in FIG. 5.

In various embodiments, the schematic diagram in FIG. 4 is considered a simplified version of the architecture for the PK submodule 302. For example, this schematic diagram includes at least a portion of the various components (e.g., inputs, logical units, mathematical components, neural network layers, etc.) included in the PK submodule 302. In various embodiments, one or more additional components may be included in the PK submodule 302. For example, one or more additional components may be used to transform or otherwise process one or more inputs prior to those one or more inputs being sent into the catenation unit 402. Similarly, one or more additional components may be used to transform or otherwise process the output of any one or more components of the PK submodule 302 (e.g., the set of layers 404) to form the final current PK state 310.

FIG. 5 is a different schematic diagram of the inputs and outputs of the PK submodule 302 in accordance with various embodiments. The PK submodule 302 is included within the ODE module 201 of the neural network system 200 in FIGS. 2-3. In this embodiment, the architecture of the neural network system 200 further includes the dose input 244, a padding unit 502, a sum submodule 504, a multiplier submodule 506, a sum submodule 508, and a rectified linear unit (ReLU) 510. The padding unit 502 ensures that the dose input 244 is converted into a vector form that can be added to the previous PK state 306.

In various embodiments, an assumption is made that the previous PK state 306 includes a dose-based component (or parameter) and a non-dose-based component (or parameter). The padding unit 502 ensures that the dose input 244 has a vector form such that when the dose input 244 is added to the previous PK state 306, the dose input 244 is added to the dose-based component and the non-dose-based component remains unaffected. This adding is performed by the sum submodule 504, which outputs a revised PK state 512.

The revised PK state 512 and the PK vector 220 are input into the PK submodule 302 (or a portion of the PK submodule 302). The PK submodule 302 (or the portion of the PK submodule 302) outputs an initial state change vector 514. The multiplier submodule 506 multiplies the Euler time step by the initial state change vector 514 to produce a new state change vector 516 that is then added to the revised PK state 512 by the sum submodule 508. The ReLU 510 takes the output from the sum submodule 508 and produces the current PK state 310 having no negative values.

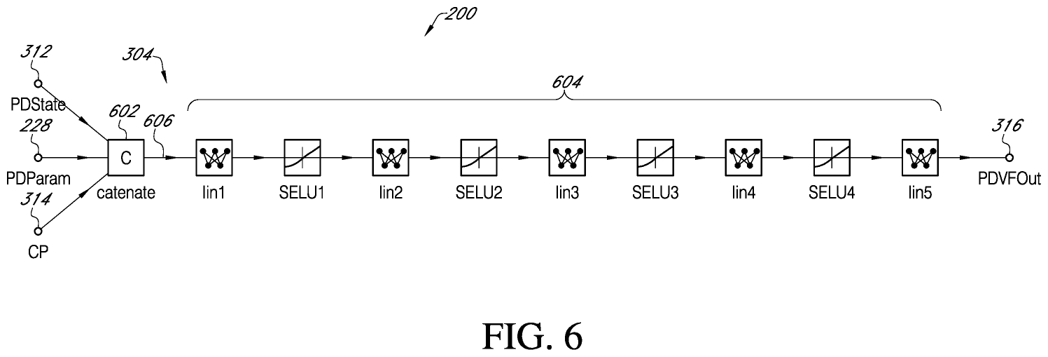

FIG. 6 is a schematic diagram of the PD submodule 304 from FIG. 3 in accordance with various embodiments. The PD submodule 304 is included within the ODE module 201 of the neural network system 200 in FIGS. 2-3. The PD submodule 304 includes a catenation unit 602 and a set of layers 604 that process the output of the catenation unit 602 to then generate the current PD state 316. The catenation unit 602 combines the previous PD state 312 with the PD vector 228 to generate a PD processing vector 606 that is transformed via the set of layers 604. The set of layers 604 may include any number or type of layers including, but not limited to, linear layers and Scaled Exponential Linear Unit (SELU) layers. In one or more examples, the set of layers 604 includes 5 linear layers and 4 SELU layers, as shown in FIG. 6.

In various embodiments, the schematic diagram in FIG. 6 is considered a simplified version of the architecture for the PD submodule 304. For example, this schematic diagram includes at least a portion of the various components (e.g., inputs, logical units, mathematical components, neural network layers, etc.) included in the PD submodule 304. In various embodiments, one or more additional components may be included in the PD submodule 304. For example, one or more additional components may be used to transform or otherwise process one or more inputs prior to those one or more inputs being sent into the catenation unit 402. Similarly, one or more additional components may be used to transform or otherwise process the output of any one or more components of the PD submodule 304 (e.g., the set of layers 604) to form the final current PD state 316. The final current PD state 316 is the PD vector field (PDVF) output (e.g., PDVFOut).

II.B.3. Exemplary Input Data for Neural Network System

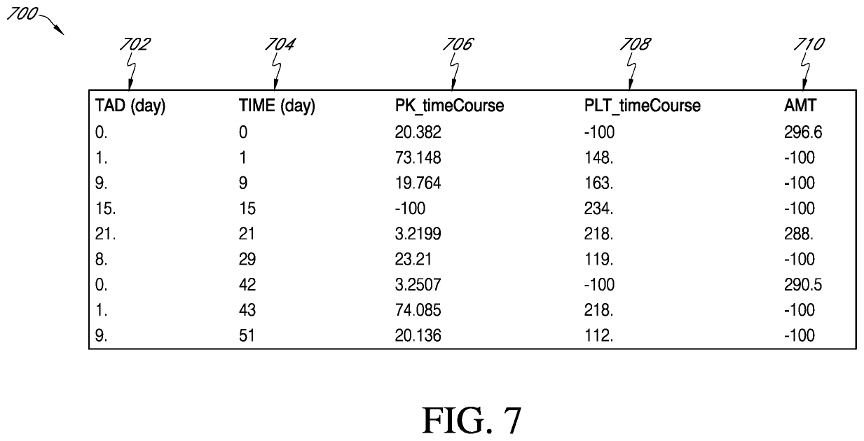

FIG. 7 is a portion of a table of values used as input data in accordance with various embodiments. The table 700 is an example of one table that is used in the input data 202 described with respect to FIG. 2. The table 700 includes the following columns: time-after-dose 702, time 704, dose effect (e.g., drug concentration) 706, drug effect (e.g., biomarker effect) 708, and dose amount 710. A value for time-after-dose 702 is the time after the administration of a drug dose in days. In other embodiments, the time may be measured in hours, minutes, or some other unit of time. A value for time 704 is the time with respect to the beginning or initial state of the study or clinical trial in days. In one example, the beginning or initial state of a trial may be considered day zero. In other embodiments, the time may be measured in hours, minutes, or some other unit of time. A value for dose effect 706 identifies the systemic concentration of the drug, which may be in micrograms per milliliter (μg/mL). A value for drug effect 708 identifies a biomarker count, which in this case is platelet count, per liter (e.g., 10⁹/L).

While nine rows are shown, the table 700 may include any number of rows. Each row contains information for a different point in time for a particular subject.

In various embodiments, multiple tables, each being similar to table 700 and each being for a different subject are used to form the input data 202. For example, the input data 202 may be formed from n tables for n subjects, with each of the tables including data for a time period, t. The n may be selected from, for example, but is not limited to, a number between 25 and 1,000,000. The t may be selected from, for example, but is not limited to, 10 days, 21 days, 30 days, 45 days, 60 days, 3 months, another time period, etc. In other embodiments, the input data 202 is formed using a single table, similar to 700, that includes data for multiple subjects for a given time period.

IIC. Exemplary Methodologies Associated with PK/PD Modeling

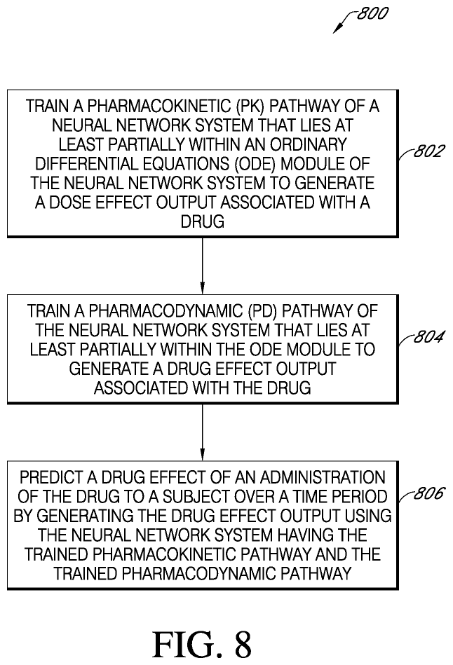

FIG. 8 is a flowchart of a process 800 for training a neural network system and using the trained neural network system to predict pharmacokinetic-pharmacodynamic effects over time in accordance with various embodiments. The process 800 may be implemented using the PK/PD evaluation system 100 in FIG. 1. Further, this process 800 may be implemented using a neural network system, such as the neural network system 110 in FIG. 1 and/or the neural network system 200 in FIG. 2.

Step 802 includes training a pharmacokinetic (PK) pathway of a neural network system that lies at least partially within an ordinary differential equations (ODE) module of the neural network system to generate a dose effect output associated with a drug. In various embodiments, the PK pathway includes a PK encoder and a PK submodule, the PK submodule being part of the ODE module of the neural network system. The PK pathway may be, for example, the PK pathway 206 in FIG. 2. The PK encoder is trained to generate a PK vector and the PK submodule is trained to use the PK vector, dosing, and an initial condition to generate a dose effect output. The dosing may be provided from the training data, may be at least partially extrapolated from the training data, may be input by a user, or some combination thereof. The initial condition for the PK submodule is typically zero. The PK encoder determines the “meaning” or “context” of the parameters in the PK vector. In one or more examples, at least one of the parameters incorporates the dosing.

Step 804 includes training a pharmacodynamic (PD) pathway of the neural network system that lies at least partially within the ODE module to generate a drug effect output associated with the drug. In various embodiments, the PD pathway includes a PD encoder and a PD submodule, the PD submodule being part of the ODE module of the neural network system. The PD pathway may be, for example, the PD pathway 208 in FIG. 2. The PD encoder is trained to generate a PD vector and the PD submodule is trained to use the PD vector, the dose effect output from the PK pathway, and an initial condition. In the various embodiments, at a given time step during the training, the PD submodule uses the dose effect value for the immediately preceding time step, along with the PD vector, to determine the drug effect value for that given time step. In the various embodiments, the initial condition for the PD submodule is provided via an IC pathway within the neural network system that is trained simultaneously with the PD pathway.

Step 806 includes predicting a drug effect of an administration of the drug to a subject over a time period by generating the drug effect output suing using the neural network system having the trained pharmacokinetic pathway and the trained pharmacodynamic pathway. While steps 802 and 804 use the neural network system in a training mode, step 806 involves using the neural network system in a prediction mode. The drug effect output is dependent on the dose effect output, which is in turn, depending on dosing. Accordingly, step 806 also may include predicting a dose effect of the administration of the drug to the subject over the time period by generating the dose effect output using the trained pharmacokinetic pathway. The drug effect output is a drug effect time course. For example, the ODE module may output a biomarker effect (e.g., platelet count, neutrophil count, tumor cell count, etc.) over time in the form of a continuous function.

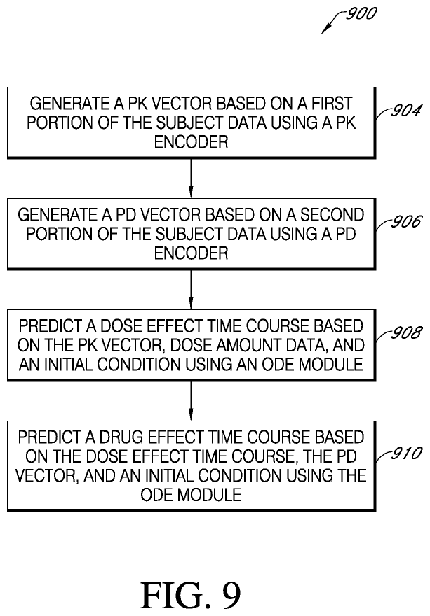

FIG. 9 is a flowchart of a process 900 for predicting pharmacokinetic-pharmacodynamic effects over time in accordance with various embodiments. The process 900 may be implemented using the PK/PD evaluation system 100 in FIG. 1. Further, this process 900 may be implemented by one or more processors using a neural network system, such as the neural network system 110 in FIG. 1 and/or the neural network system 200 in FIG. 2.

Step 902 includes receiving subject data for an initial time period. The subject data may be, for example, data that tracks dose effect and drug effect over arbitrary points in time within the initial time period for a plurality of subjects (e.g., patients). The initial time period may be, for example, a set number of hours, days, etc. In one or more examples, the initial time period is selected as a time period between 5 days and 5000 days.

Step 904 includes generating a PK vector based on a first portion of the subject data using a PK encoder. This first portion includes, for example, time-after-dose values, time values, dose effect values, and dose amount values for the initial time period. The PK encoder in the neural network system may include, for example, a set of GRUs.

Step 906 includes generating a PD vector based on a second portion of the subject data using a PD encoder. The second portion includes, for example, time-after-dose values, time values, dose effect values, and drug effect values for the initial time period. Of note, dose amount values are not needed by the PD encoder. The PD encoder in the neural network system may include, for example, a set of GRUs.

Step 908 includes predicting a dose effect time course based on the PK vector, dose amount data, and an initial condition using an ODE module. The initial condition for dose effect is always zero as there is no effect on the drug in the body prior to the drug being administered to the body. The dose amount data may include at least a portion of the dose amount values from the subject data. In various embodiments, the dose amount data also includes additional dose amount values provided by some other source (e.g., an operator, a technician, a medical professional, the one or more processors, etc.). For example, the subject data may include dose amount values for the arbitrary time points within the initial time period. Additional dose amount values may be supplemented to fill in certain time points within the initial time period and/or the time points after the initial time period. The dose effect time course is predicted by a PK submodule of the ODE module, the PK submodule including a set of GRUs, a set of ODE solvers, or a combination thereof.

Step 910 includes predicting a drug effect time course based on the dose effect time course, the PD vector, and an initial condition using the ODE module. The initial condition (IC) is generated by adjusting an initial state extracted from the subject data with an IC correction provided by an IC submodule in the neural network system. The drug effect time course is predicted by a PD submodule of the ODE module, the PD submodule including a set of GRUs, a set of ODE solvers, or a combination thereof. By taking into account the dose effect time course in determining the drug effect time course, the ODE module indirectly takes into account the effect of dosing on the drug effect.

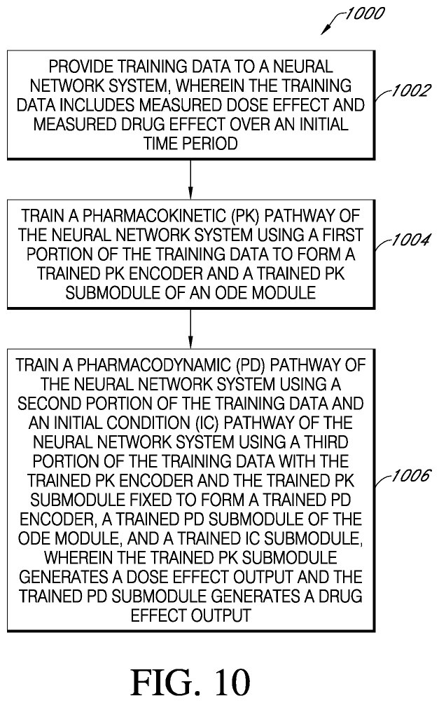

FIG. 10 is a flowchart of a process 1000 for training a neural network system to predict pharmacokinetic-pharmacodynamic effects over time in accordance with various embodiments. The process 1000 may be implemented using the PK/PD evaluation system 100 in FIG. 1. Further, this process 1000 may be implemented by one or more processors using a neural network system, such as the neural network system 110 in FIG. 1 and/or the neural network system 200 in FIG. 2.

Step 1002 includes providing training data to a neural network system, wherein the training data includes measured dose effect and measured drug effect over an initial time period. In one or more examples, the training data includes time-after-dose values, time values, dose effect values, drug effect values, and dose amount values.

In various embodiments, step 1002 is performed by receiving initial clinical data for a plurality of subjects (e.g., patients) for the initial time period and generating the training data from the initial clinical data by apportioning a plurality of training datasets from the initial clinical data. The initial time period may be, for example, but is not limited to, a number of days between 5 days and 5000 days. The number of subjects may be, for example, 200, 300, 400, 500, 750, 800, 1000, or some other number of subjects. In one example, the initial clinical data includes data for N subjects, where the data for a first portion (e.g., 75%, 80%, or another %) of the subjects (e.g., N₁ subjects) is selected for training and the data for a second portion (e.g., 25%, 20%, or another %), of the subjects (e.g., N₂ subjects) is selected for testing. The first portion of the data for the N₁ subjects may be normalized. In one or more examples, this normalization includes ensuring that each column is normalized to have a mean of zero and a standard deviation of one. The resulting scaling factors from this normalization are later used to transform the second portion of the data for the N₂ subjects used for testing. This second portion of the data for the N₂ subjects may be used at the very end of training to perform a final evaluation of the neural network system.

In these examples, the plurality of training datasets is apportioned from the first portion of the data for the N₁ subjects, with each of the plurality of training datasets corresponding to a different portion of the initial time period. As one example, the initial time period may be 90 days. The plurality of training datasets may include m training datasets (m≥2), with each of the m training datasets including that portion of the initial clinical data up to a corresponding number of days within the 90 days:

TABLE 1
m Training Datasets,
Initial Time Period = 90 Days

| | |
|---|---|
| 1^{st} Training Dataset | 0 to 15 days |
| 2^{nd} Training Dataset | 0 to 21 days |
| 3^{rd} Training Dataset | 0 to 30 days |
| 4^{th} Training Dataset | 0 to 40 days |
| . . . | |
| . . . | |
| m^{th} Training Dataset | 0 to 90 days |

In this manner, the initial clinical data is augmented to thereby enrich the training data used to train the neural network system. This type of augmentation helps force the neural network system to achieve its goal of enabling accurate and reliable predictions based on early observation a dosing record.

Step 1004 includes training a PK pathway of the neural network system using a first portion of the training data to form a trained PK encoder and a trained PK submodule of an ODE module. The PK pathway may be, for example, the PK pathway 206 in FIG. 2. The PK pathway is trained prior to the training of the PD pathway and the IC pathway. Training the PK pathway of the neural network system before the PD pathway of the neural network system is important because the drug effect depends on the dose effect.

The PK pathway is trained using batch and/or epoch processing and backpropagation to reduce loss and/or error. For example, each of the plurality of training datasets formed may be randomly sampled and split into batches. Each batch is run through just the PK pathway of the neural network system to train the PK encoder and the PK submodule of the ODE module, with backpropagation being used to modify the weights/factors of the PK encoder and PK submodule as needed after each batch. The cycle time for passing the entire training dataset (in batches) through the PK pathway is one epoch. Multiple epochs (e.g., 100 epochs, 1000 epochs, 2000 epochs, 3000 epochs, etc.) may be used for each training dataset. The number of epochs used may be selected to arrive at the desired weights/factors and reduce underfitting and overfitting. Of course, in other embodiments, other variations of the batch and/or epoch processing may be used to train the PK pathway with the plurality of training datasets.

Backpropagation is performed by comparing the output of the neural network system, which includes a dose effect time course and a drug effect time course, to the observed data. In particular, the trainable weights of the PK encoder and the PK submodule are iteratively refined to minimize the Least Square Errors (L₂ loss) function between the output of the neural network system and the observed data.

Step 1006 includes training a PD pathway of the neural network system using a second portion of the training data and an IC pathway of the neural network system using a third portion of the training data with the trained PK encoder and the trained PK submodule fixed to thereby form a trained PD encoder, a trained PD submodule of the ODE module, and a trained IC submodule, wherein the trained PK submodule generates a dose effect output and the trained PD submodule generates a drug effect output. The PD pathway and the IC pathway may be, for example, the PD pathway 208 and the IC pathway 210, respectively, in FIG. 2. The trained IC submodule generates an IC correction vector that is used to adjust an initial condition for the ODE module. In various embodiments, this IC correction vector only affects the PK submodule's initial condition as the initial condition for the PD submodule is generally zero.

In various embodiments, the training of the PD pathway and the IC pathway includes fixing the weights associated with the PK encoder and the PK submodule of the PK pathway. In this manner, the neural network system is built using a sequential methodology. The training of the PD pathway and the IC pathway further includes running the training data through the neural network system using batch and/or epoch processing and backpropagation similar to as described above. In this manner, the PD encoder, the PD submodule of the ODE module, and the IC submodule are trained simultaneously.

### III. Computer Implemented System

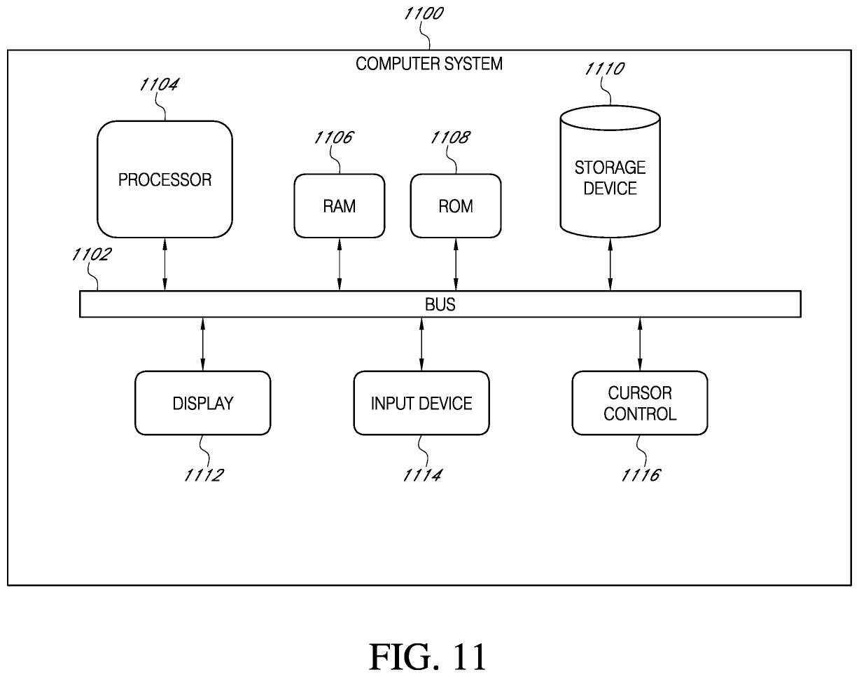

FIG. 11 is a block diagram of a computer system in accordance with various embodiments. Computer system 1100 may be an example of one implementation for computing platform 103 described above in FIG. 1. In one or more examples, computer system 1100 can include a bus 1102 or other communication mechanism for communicating information, and a processor 1104 coupled with bus 1102 for processing information. In various embodiments, computer system 1100 can also include a memory, which can be a random access memory (RAM) 1106 or other dynamic storage device, coupled to bus 1102 for determining instructions to be executed by processor 1104. Memory also can be used for storing temporary variables or other intermediate information during execution of instructions to be executed by processor 1104. In various embodiments, computer system 1100 can further include a read only memory (ROM) 1108 or other static storage device coupled to bus 1102 for storing static information and instructions for processor 1104. A storage device 1110, such as a magnetic disk or optical disk, can be provided and coupled to bus 1102 for storing information and instructions.

In various embodiments, computer system 1100 can be coupled via bus 1102 to a display 1112, such as a cathode ray tube (CRT) or liquid crystal display (LCD), for displaying information to a computer user. An input device 1114, including alphanumeric and other keys, can be coupled to bus 1102 for communicating information and command selections to processor 1104. Another type of user input device is a cursor control 1116, such as a mouse, a joystick, a trackball, a gesture input device, a gaze-based input device, or cursor direction keys for communicating direction information and command selections to processor 1104 and for controlling cursor movement on display 1112. This input device 1114 may have two degrees of freedom in two axes, a first axis (e.g., x) and a second axis (e.g., y), that allows the device to specify positions in a plane. However, it should be understood that input devices 1114 allowing for three-dimensional (e.g., x, y and z) cursor movement are also contemplated herein.

Consistent with certain implementations of the present teachings, results can be provided by computer system 1100 in response to processor 1104 executing one or more sequences of one or more instructions contained in RAM 1106. Such instructions can be read into RAM 1106 from another computer-readable medium or computer-readable storage medium, such as storage device 1110. Execution of the sequences of instructions contained in RAM 1106 can cause processor 1104 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, storage device, data storage device, etc.) or “computer-readable storage medium” as used herein refers to any media that participates in providing instructions to processor 1104 for execution. Such a medium can take many forms, including but not limited to, non-volatile media, volatile media, and transmission media. Examples of non-volatile media can include, but are not limited to, optical, solid state, magnetic disks, such as storage device 1110. Examples of volatile media can include, but are not limited to, RAM 1106 (e.g., dynamic RAM (DRAM) and/or static RAM (SRAM)). Examples of transmission media can include, but are not limited to, coaxial cables, copper wire, and fiber optics, including the wires that comprise bus 1102.

Additionally, a computer-readable medium may take various forms such as, for example, but not limited to, a floppy disk, a flexible disk, hard disk, magnetic tape, or any other magnetic medium, a CD-ROM, any other optical medium, punch cards, paper tape, any other physical medium with patterns of holes, RAM, PROM, EPROM, EEPROM, FLASH-EPROM, solid-state memory, one or more storage arrays (e.g., flash arrays connected over a storage area network), network attached storage, any other memory chip or cartridge, or any other tangible medium from which a computer can read.

In addition to computer readable medium, instructions or data can be provided as signals on transmission media included in a communications apparatus or system to provide sequences of one or more instructions to processor 1104 of computer system 1100 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, optical communications connections, etc.

It should be appreciated that the methodologies described herein, flow charts, diagrams, and accompanying disclosure can be implemented using computer system 1100 as a standalone device or on a distributed network of shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 1100, whereby processor 1104 would execute the analyses and determinations provided by these engines, subject to instructions provided by any one of, or a combination of, the memory components RAM 1106, ROM, 1108, or storage device 1110 and user input provided via input device 1114.

### IV. Examples and Results

IVA. Testing the Neural PK/PD Model Against the Existing Pop-PK/PD Model

IV.A.1. Methodology

The improved systems and methods, disclosed herein, were tested against observed data and “ground truth” data to verify their accuracy and reliability. For given initial clinical data, the ground truth data includes the portion of that clinical data not used for training. In other words, the ground truth data includes that portion of the clinical data set aside for testing.

In these examples, a neural network system (e.g., one example of an implementation for neural network system 110 in FIG. 1) was benchmarked against a pop-PK/PD model using data comprised of longitudinal platelet response for 655 patients receiving a trastuzumab emtansine (T-DM1) treatment, an approved anti-cancer therapy for treating human epidermal growth receptor 2 (HER2)-positive metastatic breast cancer. Of this data set, 80% of the patient records were used for training (the “training data set”) and 20% of the patient records were used for testing (the “testing data set”). Data normalization was performed for both the training data set and the testing data set to ensure that each parameter had values with a mean of 0 and a standard deviation of 1.

To combat overfitting, data augmentation was performed, where different augmented records were created for each patient. For example, the entire time course for a given patient i was used to construct 5 augmented records, where the neural network system would be fed an “input” and asked to predict an “output” as the prediction target (input→output):

Complete time course: for training patient i,

{PKⁱ(t), Dosingⁱ(t), PDⁱ(t)}_{0≤t<∞}→{PKⁱ(t), PDⁱ(t)}_{0≤t<∞}

Observation data up to day 21: for training patient i,

{PKⁱ(t), PDⁱ(t)}_{0≤t<21}, {Dosingⁱ(t)}_{0≤t<∞}→{PKⁱ(t), PDⁱ(t)}_{0≤t<∞}

Observation data up to day 35: for training patient i,

{PKⁱ(t), PDⁱ(t)}_{0≤t<35}, {Dosingⁱ(t)}_{0≤t<∞}→{PKⁱ(t), PDⁱ(t)}_{0≤t<∞}

Observation data up to day 42: for training patient i,

{PKⁱ(t), PDⁱ(t)}_{0≤t<42}, {Dosingⁱ(t)}_{0≤t<∞}→{PKⁱ(t), PDⁱ(t)}_{0≤t<∞}

Observation data up to day 63: for training patient i,

{PKⁱ(t), PDⁱ(t)}_{0≤t<63}, {Dosingⁱ(t)}_{0≤t<∞}→{PKⁱ(t), PDⁱ(t)}_{0≤t<∞}

This data augmentation yielded a 532×5=2660 set of augmented patient records. This type of data augmentation helped force the neural network system to achieve to the goal of enabling predictions in for other time periods.

Both the neural network system and the pop-PK/PD model were trained using the training data set. Performance was then evaluated using the testing data and compared with respect to both r squared (r2) and RMSE. The performance of the neural network system was evaluated using the testing data set for a period of time (e.g., observation window), tObs, selected from initial clinical data. This tObs was set to 21, 42, and 63 days. For each tObs, the portion of a patient's clinical data falling within the tObs (t<tObs) was used to predict all future output data (e.g., dose effect and drug effect) after the tObs (t≥tObs).

IV.A.2. Results

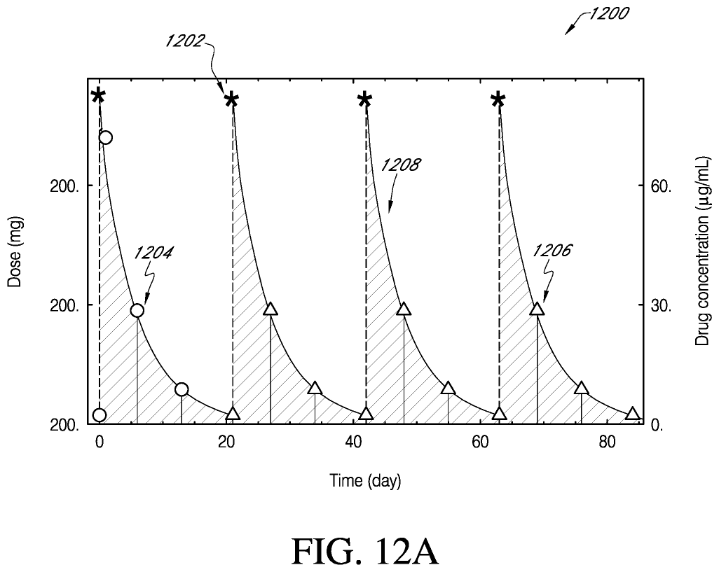

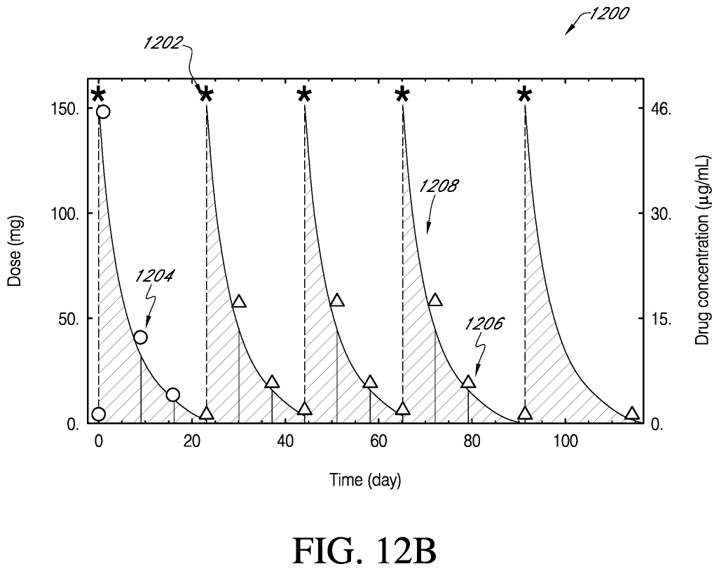

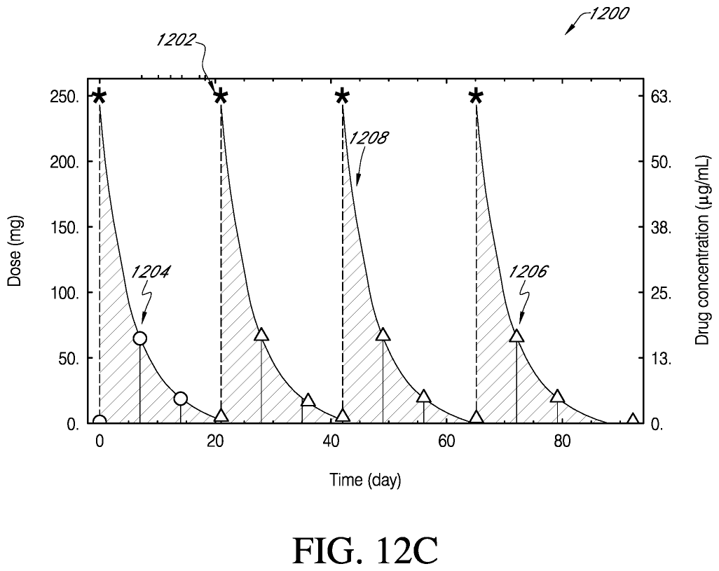

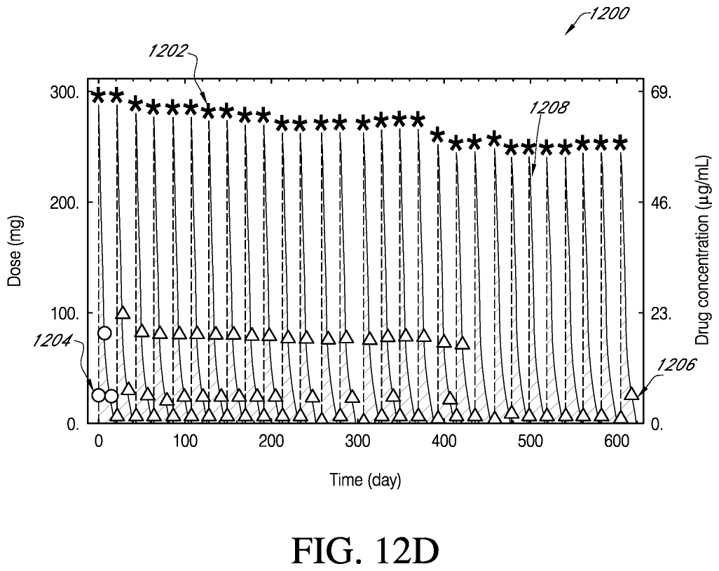

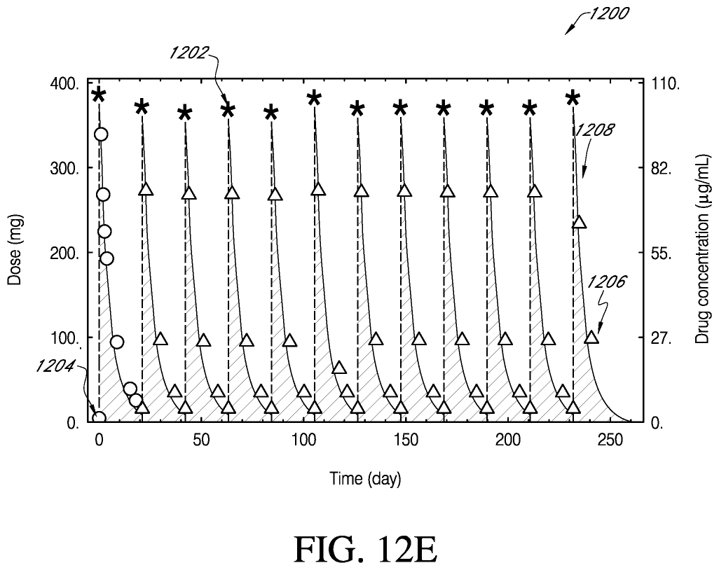

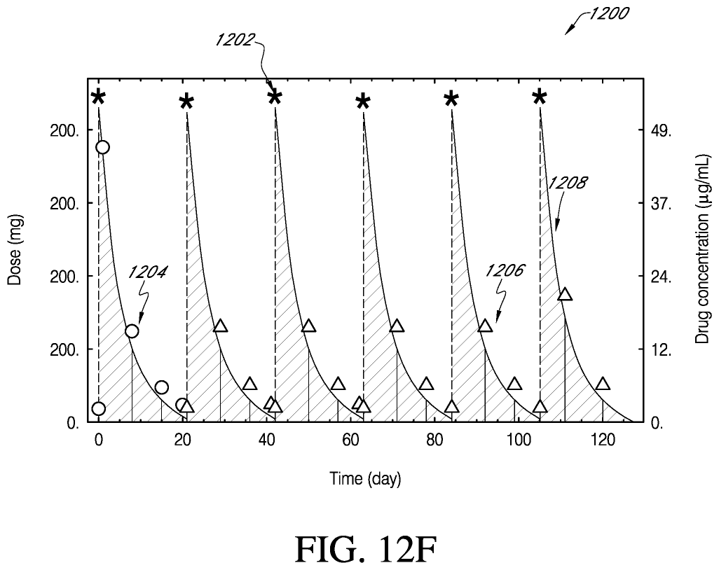

The testing shows that the neural PK/PD model of the embodiments described herein results in more precise predictions using less observation data when compared to the current “gold standard” PK/PD model. For instance, when the neural network (e.g., the ODE module 201) is limited to “searching” within a space of ODE systems (e.g., a space no larger than that of the existing pop-PK/PD model), the neural network provides an improved neural-PK/PD model that outperforms the existing pop-PK/PD model. These results indicate that neural PK/PD modeling warrants further development and validation to enable applications such as precision dosing and novel dosing regimen creation. FIGS. 12A-F, 13A-F, and 14A-F are series of plots that demonstrate the accuracy of using the neural network system of the various embodiments described herein in accordance with various embodiments.

FIGS. 12A-12F are plots in a plot series 1200 demonstrating the accuracy of the dose effect output of a neural network system in accordance with various embodiments. In particular, the plot series 1200 demonstrates the accuracy of the dose effect output generated by the neural network system 200 in FIG. 2 for six different subjects where the observation window, tObs, is set to 21 days. In this plot series 1200, the dose effect being studied is drug concentration in micrograms per milliliter (μg/mL). In each plot of the plot series 1200, the stars 1202 represent dose amount, the circles 1204 represent the drug concentration values within the observation window, the triangles 1206 represent the ground truth drug concentration values, and the curves 1208 represent the drug concentration values output from the neural network model.

As depicted in this example, the observed data exists up to and including 21 days. The ground truth data provides comparison data for the time after 21 days. The curves 1208 are generated based on training performed using the training data for the 21-day observation window. Comparing the curves 1208 to the triangles 1206 validates that the neural network system provides accurate drug concentration values beyond the observation window.

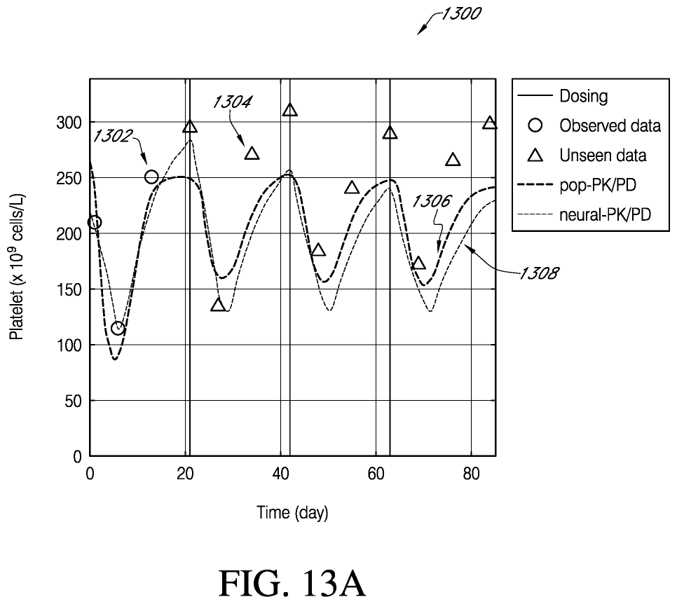

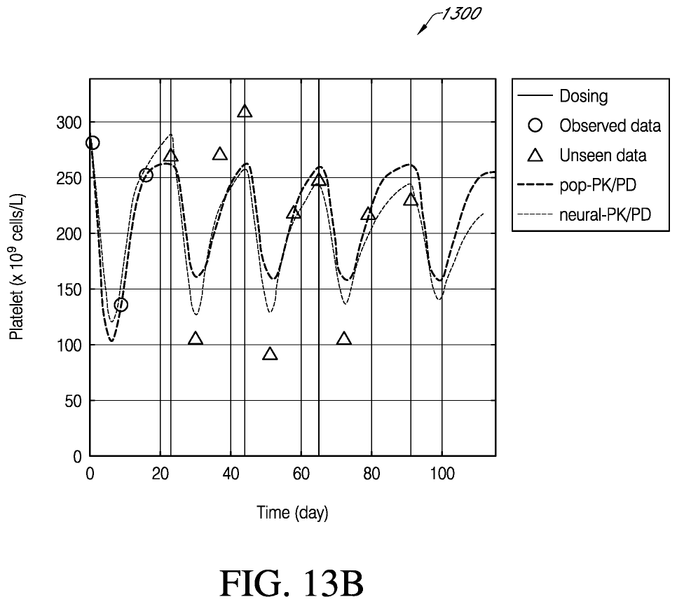

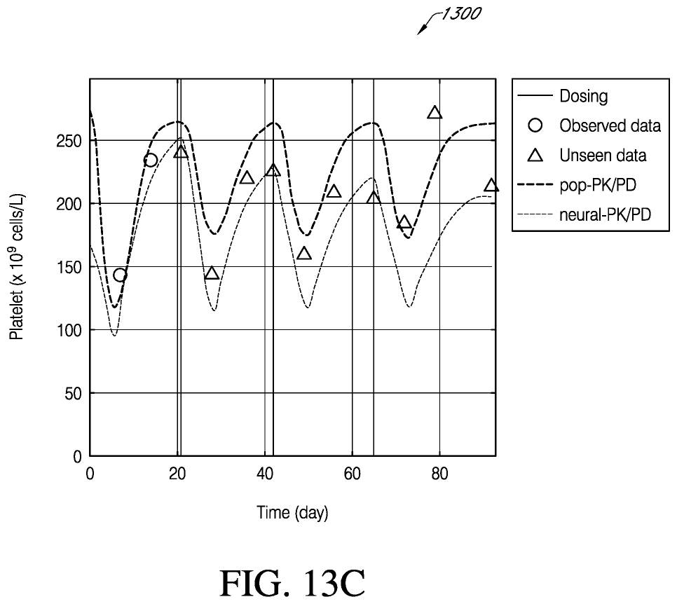

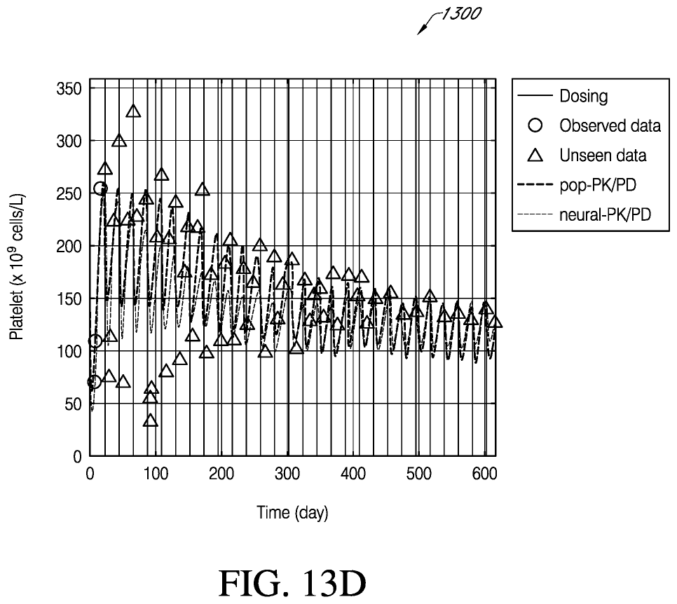

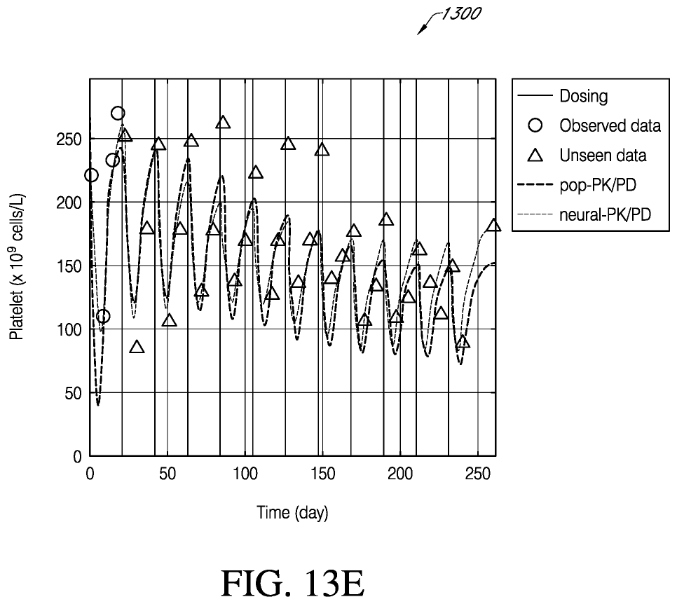

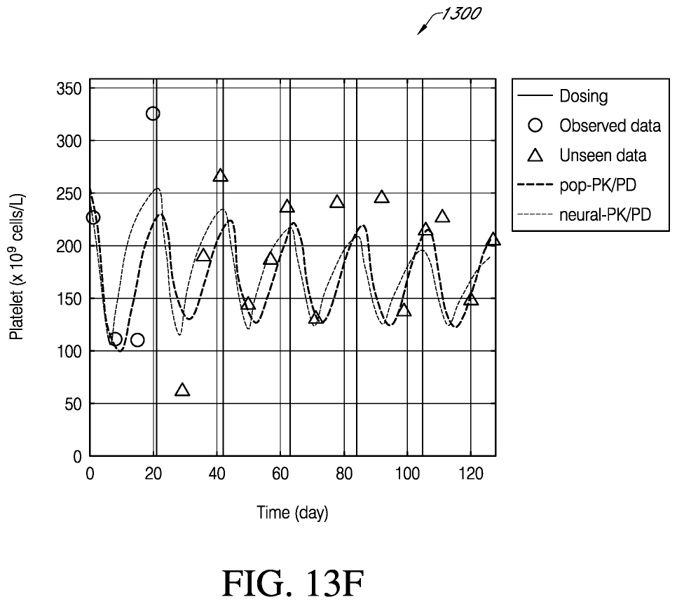

FIGS. 13A-13F are plots in a plot series 1300 demonstrating the accuracy of the drug effect output of a neural network system in accordance with various embodiments. In particular, the plot series 1300 demonstrates the accuracy of the drug effect output generated by the neural network system 200 in FIG. 2 for six different subjects. In this plot series 1300, the drug effect being studied is platelet count in cells per liter (×10⁹ cells/L). In each plot of the plot series 1300, the circles 1302 represent the platelet count observed (or measured), the triangles 1304 represent the ground truth data, the first dashed curve 1306 represents the platelet count time course generated by a population PK/PD model, and the second dashed curve 1308 represents the platelet count time course generated by the neural network system.

As depicted, the observed data exists up to 21 days. The ground truth data provides comparison data for the time period of 21 days and afterwards. Comparing the ground truth data and the platelet count time course generated by the population PK/PD model to the platelet count time course generated by the neural network system validates the ability of the neural network system to accurately predict the platelet count for the period after 21 days.

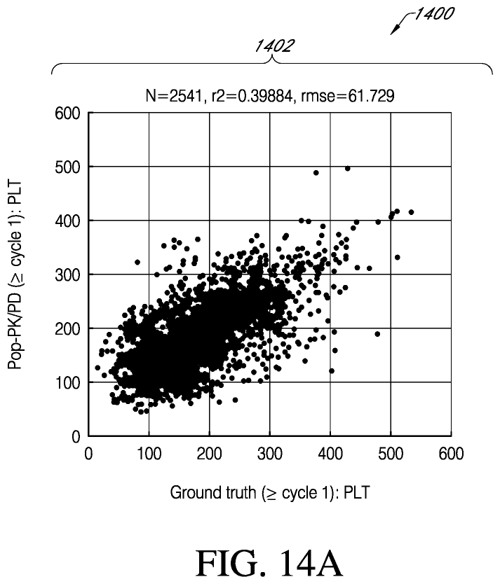

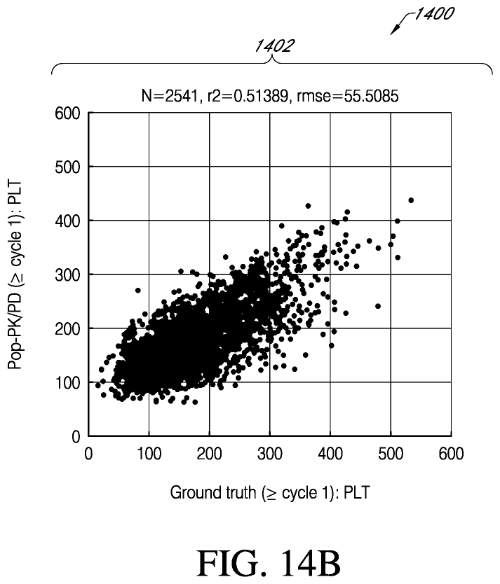

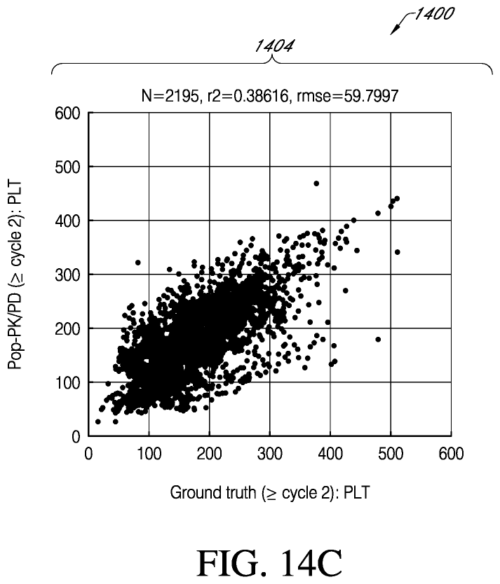

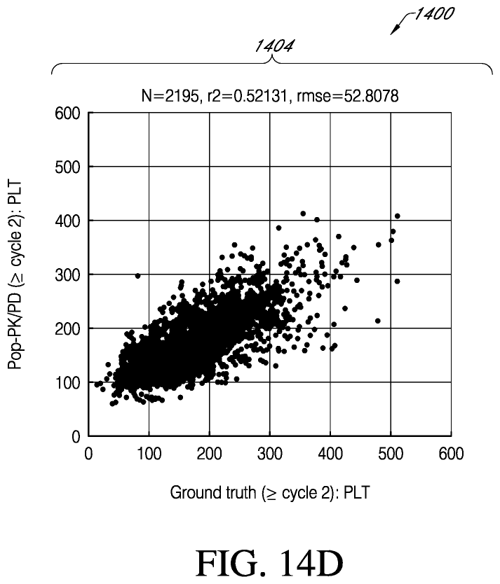

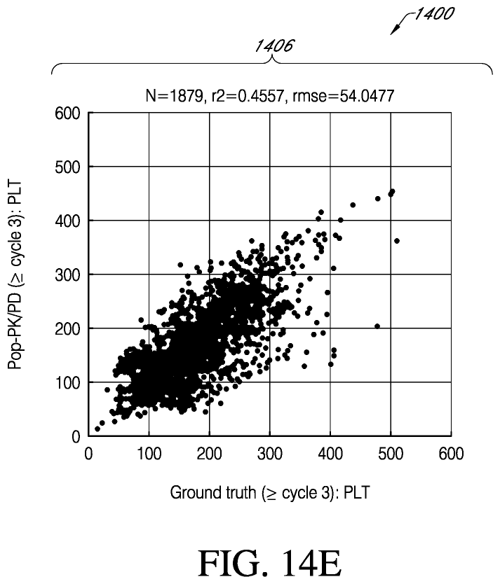

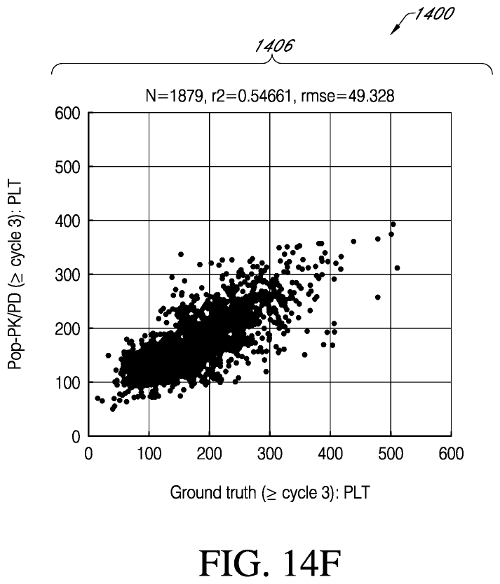

FIGS. 14A-14F are plots in another plot series 1400 demonstrating the accuracy of the neural network system in accordance with various embodiments. The plot series 1400 includes a pair of plots 1402 for a 21-day observation window (or observation time period), a pair of plots 1404 for a 42-day observation window, and a pair of plots 1406 for a 63-day observation window. Each pair of plots includes a first plot comparing the platelet count time course output from the population PK/PD model to the ground truth data and a second plot comparing the platelet count time course output from the neural network system to the ground truth data. In each plot, the N refers to the number of predictions made for the corresponding scenario. The plot series 1400 shows that the neural network system has a numerically higher r-squared (r2) value and lower root-mean-squared error (rmse) for each observation window.

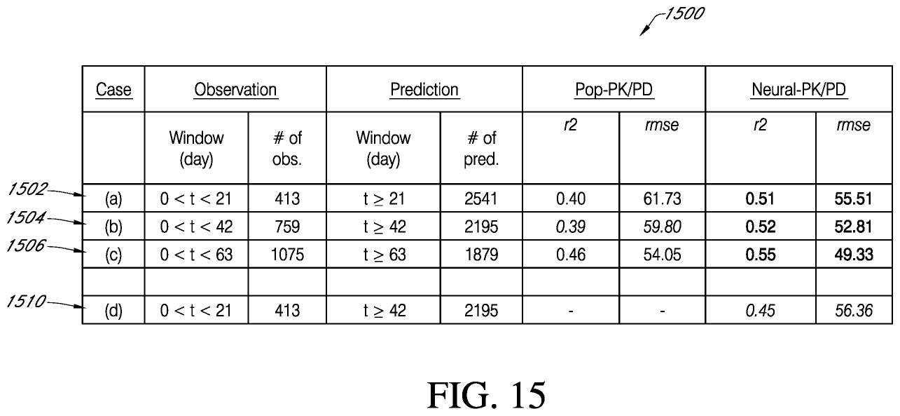

FIG. 15 is a table comparing the predictive performance of a population PK/PD model to a neural network system per the various embodiments described herein. As shown, the neural network system consistently outperforms the population PK/PD model in predictive performance. Case (a) 1502 compares the r-squared values and root-mean-squared errors for the population PK/PD model and the neural network system for an observation window up to 21 days, with 413 observations. Case (b) 1504 compares the r-squared values and root-mean-squared errors for the population PK/PD model and the neural network system for an observation window up to 42 days, with 759 observations. Case (c) 1506 compares the r-squared values and root-mean-squared errors for the population PK/PD model and the neural network system for an observation window up to 63 days, with 1075 observations. Case (d) 1508 is provided to show that the neural network model has a higher r-squared value and lower root-mean-squared error for predictions beyond 42 days, using the observation window up to and including 21 days, as compared to the population PK/PD model in case (b) 1504, which uses nearly double the number of observations for the same prediction and the neural network system for an observation window up to 42 days.

IVB. Neural PK Model Using ODE—Predictions for Untested Treatment Regimens

A neural PK model constructed according to one or more of the embodiments described herein was evaluated. The neural PK model is formed by the pharmacokinetic pathway of the neural network system (e.g., neural network system 110 in FIG. 1) described herein. The neural PK model includes the portion of the ODE module that corresponds to the pharmacokinetic pathway. Performance of the neural PK model was tested and compared to the performance of other models, including a nonlinear mixed effects (NLME) model, a light gradient boost model (GBM), and a long short-term memory (LSTM) neural network.

The data from 675 patients, which contained a total of 16, 472 records of T-DM1 dosing and PK measurements, was used. The data included two distinct dosing schedules: dosing every week (Q1 W) and dosing every three weeks (Q3 W). Patient measurements were collected for a time period ranging from 1.75 hours to about 17,000 hours. The average total treatment duration for Q1 W was 5122.62 hours, while the average total treatment duration for Q3 W was 4266.28 hours.

The neural PK model outperformed the other models when used to simulate patient responses to untested dosing regimens. With respect to untested (new) dosing regimens, the neural PK model, when trained on the Q3 W data and tested on the Q1 W data, performed better than the other models with an RMSE of 10.61, an r2 of 0.76, and a Pearson's correlation of 0.89.

Further, when used for continuous profiling of PK, the neural PK model provided smoother prediction values after each dosing. The time variable being input into the neural PK model as a continuous value may account for these smoother predictions. Additionally, when simulating hypothetical situations (e.g., where dosing is stopped in the middle of a treatment course), the neural PK model was able to detect and reflect this stop in treatment by predicting zero values after the stop of dosing. However, other models (e.g., the LSTM and Light GBM models) were unable to do the same.

Thus, the neural PK model may be used to accurately predict PK effects over time and simulate responses for known and new treatment regimens.

### V. Exemplary Descriptions of Terms

The disclosure is not limited to these exemplary embodiments and applications or to the manner in which the exemplary embodiments and applications operate or are described herein. Moreover, the figures may show simplified or partial views, and the dimensions of elements in the figures may be exaggerated or otherwise not in proportion. Section divisions (e.g., heading and/or subheadings) in the specification are for ease of review only and do not limit any combination of elements discussed.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, chemistry, biochemistry, molecular biology, pharmacology, and toxicology are described herein are those well-known and commonly used in the art.

As the terms “on,” “attached to,” “connected to,” “coupled to,” or similar words are used herein, one element (e.g., a component, a material, a layer, a substrate, etc.) can be “on,” “attached to,” “connected to,” or “coupled to” another element regardless of whether the one element is directly on, attached to, connected to, or coupled to the other element or there are one or more intervening elements between the one element and the other element. In addition, where reference is made to a list of elements (e.g., elements a, b, c), such reference is intended to include any one of the listed elements by itself, any combination of less than all of the listed elements, and/or a combination of all of the listed elements.

As used herein, the term “subject” may refer to a subject of a clinical trial, a person undergoing one or more drug (or therapeutic) treatments (e.g., anti-inflammation therapeutics, infectious disease treatment, etc.), a person being monitored for remission or recovery, a person undergoing a preventative health analysis (e.g., due to their medical history), or any other person of interest. In some cases, the terms “subject” and “patient” are used interchangeably herein.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

The term “ones” means more than one.

As used herein, the term “plurality” can be 2, 3, 4, 5, 6, 7, 8, 9, 10, or more.

As used herein, “pharmacokinetics” or “pharmacokinetic” (PK) may generally refer to the study of the fate of a drug (or other substance) administered to a living organism. For example, a PK model can track how the body of the living organism affects a specific drug (or other substance) that has been administered to the body through the mechanisms of absorption and distribution. The PK properties of a drug are affected by the route of administration and the dose.

As used herein, “pharmacodynamics” or “pharmacodynamic” (PD) may generally refer to the study of the effects of a drug (or other substance) on a living organism. For example, a PD model can track the effect of a drug (or other substance) on the body by modeling its effect on a particular biomarker. It should be understood that a PD effect (e.g., a drug effect) is also dependent on dosage and may thus be also referred to as a PK/PD effect.

As used herein, “PK/PD” may refer to both PK effects and PD effects (i.e., PK/PD effects).

As used herein, a “model” may include one or more algorithms, one or more mathematical techniques, one or more neural networks, or a combination thereof.

As used herein, an “artificial neural network” or “neural network” (NN) may refer to mathematical algorithms or computational models that mimic an interconnected group of artificial neurons that processes information based on a connectionistic approach to computation. Neural networks, which may also be referred to as neural nets, can employ one or more layers of nonlinear units to predict an output for a received input. Some neural networks include one or more hidden layers in addition to an output layer. The output of each hidden layer is used as input to the next layer in the network, i.e., the next hidden layer or the output layer. Each layer of the network generates an output from a received input in accordance with current values of a respective set of parameters. In the various embodiments, a reference to a “neural network” may be a reference to one or more neural networks.

A neural network may process information in two ways; when it is being trained it is in training mode and when it puts what it has learned into practice it is in inference (or prediction) mode. Neural networks learn through a feedback process (e.g., backpropagation) which allows the network to adjust the weight factors (modifying its behavior) of the individual nodes in the intermediate hidden layers so that the output matches the outputs of the training data. In other words, it learns by being fed training data (learning examples) and eventually learns how to reach the correct output, even when it is presented with a new range or set of inputs. Examples of various types of neural networks, include, but are not limited to: Feedforward Neural Network (FNN), Recurrent Neural Network (RNN), Modular Neural Network (MNN), Convolutional Neural Network (CNN), Residual Neural Network (ResNet), and Ordinary Differential Equations Neural Networks (neural-ODE).

With a neural-ODE, the derivative of the hidden state may be parameterized using a neural network. The neural-ODE may be capable of incorporating data for arbitrary times into a continuous time-series (or time course).

As used herein, a “time course” may refer to a continuous or near-continuous time series. For example, a time course for a particular variable may track changes to that variable over time using a continuous function or a near-continuous function.

As used herein, an “encoder” may refer to a type of neural network that learns to encode (e.g., efficiently encode) a set of data into a vector of parameters having a number of dimensions. The number of dimensions may be preselected.

As used herein, “an ordinary differential equations (ODE) module” may refer to a neural network architecture that includes at least one neural network. The at least one neural network may include, for example, at least one recurrent neural network, at least one neural-ODE solver, or a combination thereof.

### VI. Additional Considerations

Thus, the embodiments described herein provide methods and systems for predicting pharmacokinetic-pharmacodynamic effects over time. The embodiments described herein provide a novel neural PK/PD modeling framework, based on a recurrent neural network architecture, that combines the principles of PK/PD with deep learning. In particular, the embodiments described herein use neural network-derived ODEs to build PK/PD models.

In one or more embodiments, a method may include training a pharmacokinetic pathway of a neural network system that lies at least partially within an ordinary differential equations (ODE) module of the neural network system to generate a dose effect output associated with a drug. The method may further include training a pharmacodynamic pathway of the neural network system that lies at least partially within the ODE module to generate a drug effect output associated with the drug. The method may further include predicting the drug effect output associated with an administration of the drug over a time period using the neural network system.

In one or more embodiments, training the pharmacokinetic pathway includes training a pharmacokinetic encoder using pharmacokinetic training data extracted from input data to form a trained pharmacokinetic encoder that outputs a pharmacokinetic vector. In one or more embodiments, training the pharmacokinetic encoder is performed using a time-after-dose value, a time value, a dose effect value, and a dose amount value for each of a plurality of subjects extracted from the input data for an observation time period to thereby form the trained pharmacokinetic encoder that outputs the pharmacokinetic vector.

In one or more embodiments, the ODE module of the neural network system includes at least one neural-ODE solver. In one or more embodiments, the pharmacokinetic pathway includes a pharmacokinetic encoder and the pharmacodynamic pathway includes a pharmacodynamic encoder. Each of the pharmacokinetic encoder and the pharmacodynamic encoder may include a set of gated recurrent units. In one or more embodiments, a dose amount is input to (into) the ODE module.

In one or more embodiments, a method includes receiving initial subject data for an initial time period and generating a pharmacokinetic vector based on a first portion of the initial subject data using a pharmacokinetic encoder. The method may further include generating a pharmacodynamic vector based on a second portion of the initial subject data using a pharmacodynamic encoder. The method may further include predicting a dose effect output based on the pharmacokinetic vector, dose amount data, and an initial condition using an ordinary differential equations (ODE) module; and predicting a drug effect output based on the dose effect output, the pharmacodynamic vector, and an initial condition using the ODE module.

In one or more embodiments, the ODE module includes at least one neural-ODE solver. In one or more embodiments, the pharmacokinetic encoder, the pharmacodynamic encoder, and the ODE module each include at least one recurrent neural network.

In one or more embodiments, a system is provided for predicting pharmacokinetic-pharmacodynamic effects over time. The system may include a memory that comprises a machine readable medium comprising machine executable code and may further include a processor coupled to the memory. The processor may be configured to execute the machine executable code to cause the processor to: train a pharmacokinetic pathway of a neural network system that lies at least partially within an ordinary differential equations (ODE) module of the neural network system to generate a dose effect output associated with a drug; train a pharmacodynamic pathway of the neural network system that lies at least partially within the ODE module to generate a drug effect output associated with the drug; and predict the drug effect output associated with an administration of the drug over a time period using the neural network system. The dose effect output may be a drug concentration over time. The drug effect output may be a biomarker effect over time. The biomarker effect may be selected from the group consisting of a platelet count, a neutrophil count, and a tumor cell count.

In one or more embodiments, the machine executable code further causes the processor to train an initial condition pathway of the neural network system simultaneously with the pharmacodynamic pathway to generate an initial condition for a pharmacodynamic submodule of the ODE module. In one or more embodiments, the pharmacokinetic pathway is trained prior to the pharmacodynamic pathway. In one or more embodiments, the pharmacodynamic pathway and the initial condition pathway are trained simultaneously.

In one or more embodiments, the pharmacokinetic pathway includes a pharmacokinetic encoder that generates a pharmacokinetic vector and a pharmacokinetic submodule of the ODE module that uses the pharmacokinetic vector to generate the dose effect output. In one or more embodiments, the pharmacodynamic pathway includes a pharmacodynamic encoder that generates a pharmacodynamic vector and a pharmacodynamic submodule of the ODE module that uses the pharmacodynamic vector and the dose effect output to generate the drug effect output.

In one or more embodiments, the pharmacokinetic pathway and the pharmacodynamic pathway are trained using training data that includes, for each of a plurality of subjects, time-after-dose values, time values, dose effect values, drug effect values, and dose amount values. In one or more embodiments, the pharmacokinetic pathway includes a pharmacokinetic encoder that generates a pharmacokinetic vector using at least a portion of the training data. In one or more embodiments, the pharmacodynamic pathway includes a pharmacodynamic encoder that generates a pharmacodynamic vector using at least a portion of the training data. In one or more embodiments, the pharmacokinetic pathway includes a pharmacokinetic submodule in the ODE module that includes at least one neural-ODE solver, the pharmacodynamic pathway includes a pharmacodynamic submodule in the ODE module that includes at least one neural-ODE solver, or both.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications, alternatives, and equivalents are possible within the scope of the invention claimed. Thus, it should be understood that although the present invention as claimed has been specifically disclosed by embodiments and optional features, modifications, variations, and/or equivalents of the concepts herein disclosed may be resorted to by those skilled in the art, and that such modifications, variations, and/or equivalents are considered to be within the scope of this invention as defined by the appended claims.

The description provides preferred exemplary embodiments only, and is not intended to limit the scope, applicability, or configuration of the disclosure. Rather, the description of the preferred exemplary embodiments will provide those skilled in the art with an enabling description for implementing various embodiments. It is understood that various changes may be made in the function and arrangement of elements without departing from the spirit and scope as set forth in the appended claims. For example, in describing the various embodiments, the specification may have presented a method and/or process as a particular sequence of steps. However, to the extent that the method or process does not rely on the particular order of steps set forth herein, the method or process should not be limited to the particular sequence of steps described, and one skilled in the art can readily appreciate that the sequences may be varied and still remain within the spirit and scope of the various embodiments.

Specific details may be provided to provide a thorough understanding of the embodiments. However, it will be understood that the embodiments may be practiced without these specific details. For example, circuits, systems, networks, processes, or other components may be shown as components in block diagram form in order not to obscure the embodiments in unnecessary detail. In other instances, well-known circuits, processes, algorithms, structures, or techniques may be shown without unnecessary detail in order to avoid obscuring the embodiments.

What is claimed is:

1. A method for predicting pharmacokinetic-pharmacodynamic effects over time, the method comprising:
training a pharmacokinetic pathway of a neural network system that lies at least partially within an ordinary differential equations (ODE) module of the neural network system to generate a dose effect output associated with a drug;
training a pharmacodynamic pathway of the neural network system that lies at least partially within the ODE module to generate a drug effect output associated with the drug; and
predicting a drug effect of an administration of the drug to a subject over a time period by generating the drug effect output suing using the neural network system having the trained pharmacokinetic pathway and the trained pharmacodynamic pathway.

2. The method of claim 1, further comprising:
predicting a dose effect of the administration of the drug to the subject over the time period by generating the dose effect output using the trained pharmacokinetic pathway.

3. The method of claim 1, further comprising:
providing, by one or more processors, training data, wherein the training data includes measured dose effect and measured drug effect over an observation time period.

4. The method of claim 1, wherein training the pharmacokinetic pathway comprises:
training a pharmacokinetic encoder using pharmacokinetic training data extracted from input data to form a trained pharmacokinetic encoder that outputs a pharmacokinetic vector.

5. The method of claim 4, wherein training the pharmacokinetic pathway further comprises:
training a pharmacokinetic submodule of the ODE module using the pharmacokinetic vector to form a trained pharmacokinetic submodule configured to generate a pharmacokinetic state for each of a plurality of time steps; and
decoding the pharmacokinetic state for each of the plurality of time steps to produce a dose effect time course.

6. The method of claim 1, wherein training the pharmacodynamic pathway comprises:
training a pharmacodynamic encoder using pharmacodynamic training data extracted from input data to form a trained pharmacodynamic encoder that outputs a pharmacodynamic vector.

7. The method of claim 6, wherein training the pharmacodynamic encoder comprises:
training the pharmacodynamic encoder using a time-after-dose value, a time value, a dose effect value, and a drug effect value for each of a plurality of subjects extracted from the input data to form the trained pharmacodynamic encoder that outputs the pharmacodynamic vector.

8. The method of claim 7, wherein training the pharmacodynamic pathway further comprises:
training a pharmacodynamic submodule of the ODE module using the pharmacodynamic vector and the dose effect output to form a trained pharmacodynamic submodule configured to generate a pharmacodynamic state for each of a plurality of time steps; and
decoding the pharmacodynamic state for each of the plurality of time steps to produce a drug effect time course.

9. The method of claim 8, wherein predicting the drug effect output comprises:
predicting a biomarker effect associated with the administration of the drug over the time period.

10. The method of claim 1, further comprising:
training an initial condition pathway of the neural network system to generate an initial condition for the ODE module.

11. The method of claim 10, wherein training the initial condition pathway comprises:
training an initial condition submodule of the neural network system using input data to form a trained initial condition submodule that generates an initial condition correction vector for use in adjusting an initial state for a pharmacodynamic submodule of the ODE module.

12. The method of claim 1, further comprising:
receiving initial clinical data for a plurality of subjects for a time period; and
generating a plurality of training datasets from the initial clinical data, each of the plurality of training datasets corresponding to a different portion of the time period, to thereby form training data for use in training the pharmacokinetic pathway and the pharmacodynamic pathway.

13. A method for training a pharmacokinetic/pharmacodynamic neural network system, the method comprising:
providing training data, wherein the training data includes measured dose effect and measured drug effect over an initial time period;
training a pharmacokinetic pathway of a neural network system using a first portion of the training data to form a trained pharmacokinetic encoder and a trained pharmacokinetic submodule of an ordinary differential equations (ODE) module in the neural network system; and
training a pharmacodynamic pathway of the neural network system using a second portion of the training data and an initial condition pathway of the neural network system using a third portion of the training data with the trained pharmacokinetic encoder and the trained pharmacokinetic submodule fixed to thereby form a trained pharmacodynamic encoder, a trained pharmacodynamic submodule of the ODE module, and a trained initial condition submodule, wherein the trained pharmacokinetic submodule generates a dose effect output and the trained pharmacodynamic submodule generates a drug effect output.

14. The method of claim 13, wherein providing the training data comprises:
receiving initial clinical data for a plurality of subjects for the initial time period; and
generating the training data from the initial clinical data, wherein the training data includes a plurality of training datasets apportioned from the initial clinical data, each of the plurality of training datasets corresponding to a different portion of the initial time period.

15. The method of claim 13, wherein training the pharmacokinetic pathway comprises:
training a pharmacokinetic encoder to generate a pharmacokinetic vector; and
training a pharmacokinetic submodule of the ODE module using the pharmacokinetic vector to generate a pharmacokinetic state for each of a plurality of time steps; and
decoding the pharmacokinetic state for each of the plurality of time steps to produce a dose effect time course.

16. The method of claim 15, wherein training the pharmacodynamic pathway comprises:
training a pharmacodynamic encoder to generate a pharmacodynamic vector; and
training a pharmacodynamic submodule of the ODE module using the pharmacodynamic vector to generate a pharmacodynamic state for each of a plurality of time steps; and
decoding the pharmacodynamic state for each of the plurality of time steps to produce a drug effect time course.

17. The method of claim 13, wherein the dose effect output is a drug concentration time course and wherein the drug effect output is a biomarker effect time course.

18. A method for predicting pharmacokinetic-pharmacodynamic effects over time, the method comprising:
receiving initial subject data for an initial time period;
generating a pharmacokinetic vector based on a first portion of the initial subject data using a pharmacokinetic encoder;
generating a pharmacodynamic vector based on a second portion of the initial subject data using a pharmacodynamic encoder;
predicting a dose effect output based on the pharmacokinetic vector, dose amount data, and an initial condition using an ordinary differential equations (ODE) module; and
predicting a drug effect output based on the dose effect output, the pharmacodynamic vector, and an initial condition using the ODE module.

19. The method of claim 18, wherein:
generating the pharmacokinetic vector comprises generating the pharmacokinetic vector using time-after-dose values, time values, dose effect values, and at least one of dose amount values or drug effect values; and
generating the pharmacodynamic vector comprises generating the pharmacodynamic vector using time-after-dose values, time values, dose effect values, and drug effect values.

20. The method of claim 18, wherein:
predicting the dose effect output comprises predicting a drug concentration time course; and
predicting the drug effect output comprises predicting a biomarker effect time course.
